(19) United States
(12) Patent Application Publication
(10) Pub. No.: US 2004/0048900 A1
(43) Pub. Date: Mar. 11, 2004

(54) Nicotine and/or nicotine agonists for the treatment of general anesthetic effects and side effects

(76) Inventor: Pamela Flood, Closter, NJ (US)

Correspondence Address: Cooper & Dunham LLP, 1185 Avenue of the Americas, New York, NY, 10036, US

(21) Appl. No.: 10/464,167

(22) Filed: Jun. 17, 2003

Related U.S. Application Data
(60) Provisional application No. 60/389,690, filed on Jun. 17, 2002.

(51) Int. Cl. A61K 31/4439

(52) U.S. Cl. 514/343

(57) ABSTRACT

This invention provides a method for reducing hyperalgesia following a pain-inducing procedure being performed on a subject which comprises administering to the subject an anesthetic which is an antagonist of the subject's nicotinic acetylcholine receptor, in an amount effective to inhibit the subject's perception of pain during the pain-inducing procedure; then performing the pain-inducing procedure on the subject; and administering to the subject a hyperalgesia-reducing amount of an agonist of the subject's nicotinic acetylcholine receptor such that the subject's hyperalgesia following the procedure is reduced. This invention also provides related methods and articles of manufacture.

This application claims priority of U.S. Provisional Application No. 60/389,690, filed Jun. 17, 2002, the contents of which are hereby incorporated by reference into this application.

This invention was made with funding from the United States National Institute of General Medical Sciences Award Number K08-00695. Accordingly the United States Government has certain rights in this invention.

Throughout this application, various publications are referenced. Full bibliographic citations for these publications are found at the end of the specification immediately preceding the claims. The disclosures of these publications in their entireties are hereby incorporated by reference into this application in order to more fully describe the state of the art known to those skilled therein as of the date of the invention described and claimed herein.

### BACKGROUND OF THE INVENTION

General anesthetic drugs have a biphasic effect on pain threshold. They are hyperalgesic at very low concentrations while some provide analgesia at higher but still sub-anesthetic concentrations (Dundee, 1960; and Zhang, 2000). Many general anesthetic drugs increase pain sensitivity at the low concentrations that may be present on emergence from anesthesia. The general anesthetic used for surgery can worsen pain postoperatively.

A Volatile Anesthetic can Make Pain Worse

Emergence from general anesthesia after surgery is typically accompanied by postoperative pain. After most surgery, pain is expected and of course requires treatment. What is unexpected, and not widely known, is that the residual of many anesthetics can make pain worse. As early as 1960, Dundee showed that after emergence from a volatile anesthetic subjects had more pain sensitivity than before the anesthetic. In the same series of studies, he also showed that increased pain sensitivity was mimicked when patients were tested while breathing low concentrations of volatile anesthetics prior to the onset of their surgery (Dundee, 1960). Dundee and his colleagues went on to show that hyperalgesia (increased sensitivity to pain) is also a common response to low concentrations of many anesthetic drugs including other volatile anesthetics, barbiturates and the steroid based anesthetics (Arora, 1972; Bovill, 1971; and Briggs, 1982). All of these anesthetics are nicotinic antagonists in a clinically relevant concentration range (Flood, 1997; Violet, 1997; Paradiso, 2000; Flood, 2000; and Coates, 2001). One of the few anesthetics that Dundee found had no hyperalgesic effect, propofol, is also not a nicotinic antagonist at clinically used concentrations (Flood, 1997; and Violet, 1997). Taken together these findings suggest that nicotinic inhibition may play a role in the hyperalgesic actions of general anesthetic drugs. Increased pain sensitivity on anesthetic emergence is certainly undesirable in the face of postoperative pain. It is widely agreed that postoperative pain is inadequately treated. In 1992, the Agency for Health Care Policy and Research (AHCPR), U.S. Department of Health and Human Services, issued guidelines, “Acute Pain Management: Operative or Medical Procedures and Trauma, Clinical Practice Guideline”. These guidelines noted the widespread inadequacy of pain management and noted that unrelieved postoperative pain contributes to patient discomfort, longer recovery periods, and higher health-care costs (Panel, 1992). The pharmacological arsenal for the treatment of postoperative pain is limited largely to opiates and non-steroidal anti-inflammatory drugs. Both classes of drugs are limited in their utility by their side effects. The etiology of increased pain sensitivity in the presence of low concentrations of general anesthetics is currently unknown. Understanding the mechanism by which residual general anesthetics increase pain after surgery should open up new pathways for the treatment of postoperative pain that will be complementary to those already in place and make the post operative period a more comfortable, safer and less stressful experience.

An Animal Model for Volatile Anesthetic Hyperalgesia

Both rats and mice have been used as an animal model to study the hyperalgesic effect of volatile anesthetic drugs (Zhang, 2000; and Kingery, 2002). Volatile anesthetics cause a biphasic nociceptive response with hyperalgesic effects at the lowest concentrations and analgesic effects at higher but still subanesthetic concentrations (Zhang, 2000). In fact, a hyperalgesic phase is common to all volatile anesthetics that have been studied (Zhang, 2000). The etiology of the hyperalgesic action of volatile anesthetics is unknown.

Nicotinic Acetylcholine Receptors (nAChRs) Modulate Volatile Anesthetic Hyperalgesia

The volatile anesthetics modulate several putative targets at clinically relevant concentrations, but few of those targets are significantly affected by isoflurane concentrations as low as 0.1% (approximately 30 μM in solution at room temperature) that cause hyperalgesia (reviewed by Franks, 1998, and Flood, 1998). The mechanisms by which isoflurane acts as an analgesic and anesthetic are unknown. However, a current hypothesis suggests that isoflurane acts by inhibiting synaptic transmission. This may result from the modulation of the function of ligand gated ion channels (Franks, 1994; and Harrison, 1998). Isoflurane modulates GABA_{A}, glycine, glutamate and nAChRs at clinically relevant concentrations (Lin, 1992; Carla, 1992; Harrison, 1993; Hall, 1994; Dildy-Mayfield, 1996; Downie, 1996; and Minami, 1998). Nonetheless, despite ample evidence for modulation of the above ion channels at appropriate anesthetic concentrations, a link between modulation of specific ion channels and anesthetic induced behavior has not been established.

The low concentrations of isoflurane discussed above are below the threshold for potentiation of even the well-known GABA_{A} receptor (Wakamori, 1991; and Hall, 1994). The activation of heteromeric nicotinic receptors is inhibited by isoflurane and other volatile anesthetics at these low concentrations (Zhang, 2000; Violet, 1997; and Cardoso, 1999), suggesting that nAChR inhibition might play a role in the hyperalgesic action of isoflurane.

Nicotinic acetylcholine receptors (nAChRs) are the most potently modulated target of inhaled anesthetics (i.e., they are blocked at the lowest multiple of median alveolar concentration (MAC) (Harrison, 1998). Their inhibition occurs at concentrations well below MAC. The IC₅₀ values for the inhibition of heteromeric nAChRs by isoflurane are between 0.2 and 0.3 MAC (Flood, 1997; and Violet, 1997). Nicotine and other nicotinic agonists can act.as analgesic drugs (FIG. 15). Systemic administration of nicotine and other more potent nicotinic agonists results in potent, efficacious non-opioid analgesia (Bannon, 1998). Epibatidine, a nicotinic agonist is approximately 200 times as potent as morphine for analgesia (Qian, 1993; and Badio, 1994).

Nicotinic Acetylcholine Receptors (nAChRs)

Nicotinic acetylcholine receptors are expressed throughout the brain and spinal cord, as well as in autonomic and peripheral neurons where they both mediate synaptic transmission and act pre-synaptically to control the release of other neurotransmitters (Woolf, 1991; McGehee, 1995a; and MacDermott, 1999). Biochemical and pharmacological studies have demonstrated that there are multiple functional subtypes of nicotinic receptors present in the human brain. Nicotinic acetylcholine receptors are composed of a combination of α and β subunits arranged in a pentameric ring. Generally the receptor is composed of three β and two a subunits. Currently nine different α subunit types and 3 different β subunit types have been identified in the brain and ganglia tissue. Selected examples of nAChRs comprised of α and β subunit combinations are listed in Table 1.

TABLE 1
Examples of Nicotinic Receptor Types

| | | | | | | | | |
|---|---|---|---|---|---|---|---|---|
| α₂β₂ | α₃β₂ | α₄β₂ | α₅β₂ | α₆β₂ | α₇β₂ | α₈β₂ | α₉β₂ | α₁₀β₂ |
| α₂β₃ | α₃β₃ | α₄β₃ | α₅β₃ | α₆β₃ | α₇β₃ | α₈β₃ | α₉β₃ | α₁₀β₃ |
| α₂β₄ | α₃β₄ | α₄β₄ | α₅β₄ | α₆β₄ | α₇β₄ | α₈β₄ | α₉β₄ | α₁₀β₄ |

Subunits α₇₋₁₀ can also form homopentameric nicotinic receptors. The receptor forms listed above are merely examples of the potential combinations of α and β subunits that can form nAChRs.

Nicotinic Agonists

Nicotine is the prototypical nAChR agonist. A number of receptor-selective nAChR agonists have been isolated, including, but not limited to, DMPP, DMAC, epibatidine (U.S. Pat. No. 6/077,846), and ABT 418 (Americ, 1994). Nicotine and nicotinic agonists have been used to treat various conditions including movement disorders, dysfunction of the central or autonomic nervous systems, neurodegenerative disorders, cardiovascular disorders, convulsive disorders, drug abuse and eating disorders.

Nicotine is commonly used on an outpatient basis for smoking cessation and in children with Tourette's. Nicotine can be administered via an intranasal route. Intranasal nicotine has its peak effect in five minutes and is dissipated in about one hour. As nicotine acts as an agonist at sympathetic ganglia, it can cause increases in heart rate and blood pressure. At a dose of 3 mg intranasally, an average increase of 7 mM of mercury in systolic blood pressure and no change in diastolic blood pressure or heart rate is observed in non-smoking volunteers (Fishbein, 2000). This level of nicotine administration has minimal hemodynamic effects and results in an arterial peak concentration of 100 μM and a steady state venous concentration of 30 μM of nicotine (Guthrie, 1999). As nicotine crosses the blood-brain-barrier, these concentrations would be expected to result in significant activation of nicotinic receptors in the brain and spinal cord.

Nicotinic Nociceptive Effects in the Brain

The analgesic action of nicotine is due to an action on nicotinic aceytylcholine receptors in the central nervous system, as opposed to the periphery. Hexamethonium, a nicotinic antagonist that does not cross the blood brain barrier, has no effect on the analgesic action of nicotine (Bitner, 1998). Nicotinic agonists can cause analgesia through actions in both the brain and spinal cord. Bulbospinal modulatory systems have been implicated in both settings. Although the net effect of systemic nicotinic agonists is analgesic, nicotinic agonists applied in the brain can have either hyperalgesic or analgesic effects (Parvini, 1993; Khan, 1994; Khan, 1996; and Gillberg, 1990). Nicotine, when administered into the mid-fourth ventricle, produces analgesia in low doses and hyperalgesia in higher doses (Rao, 1996; and Parvini, 1993). Activation of the pedunculopontine tegmental nucleus and the nucleus raphe magnus with nicotine causes analgesia that is inhibited by the administration of antagonists of α₂-adrenergic, serotonergic and muscarinic receptors to the spinal cord (Iwamoto, 1993; and Iwamoto, 1991). Intracerebroventricular injection of nicotine causes increases in the release of spinal serotonin, when measured with in vivo microdialysis (Rueter, 2000). Taken together these data suggest that intact noradrenergic, serotonergic and/or cholinergic systems contribute to nicotinic (Iwamoto, 1993; Rao, 1996; Hunt, 1998; Rueter, 2000; Bitner, 1998; Chiari, 1999; and Mitchell, 1993)(FIG. 15).

Nicotinic Nociceptive Effects in the Spinal Cord

Similarly, intrathecal injection of nicotinic agonists can cause both hyperalgesic and analgesic effects (Gillberg, 1990; Aceto, 1986; Christensen, 1990; Khan, 1998; and Damaj, 1998). When rats were treated with nicotine systemically, intracerebrally or intrathecally, the intrathecal route was the most potent in causing analgesia (Aceto, 1986). In spinal rats, intrathecal nicotine causes analgesia that was reduced by the α₂-adrenergic inhibitor yohimbine, suggesting nicotinic facilitation of norepinephrine release that stimulates postsynaptic α₂-adrenergic receptors (Christensen, 1990). In the lumbar spinal cord, slice experiments have suggested that the release of serotonin is tonically controlled by nicotinic receptors (Cordero-Erausquin, 2001). Nicotinic binding sites are found predominantly in laminae II and III of the dorsal horn of the spinal cord and are almost entirely contained in the thoracic and lumbar areas (Aceto, 1986; and Gillberg, 1988). Nicotinic acetylcholine receptors are expressed on multiple axonal terminals in the CNS, where they control the release of glutamate, acetylcholine, norepinephrine, serotonin, GABA and glycine (reviewed in MacDermott, 1999; and Poulain, 1987). The hyperalgesic effects of intrathecal nicotine are thought to be due to the facilitation of glutamate release by nicotine and the activation of postsynaptic NMDA receptors (Khan, 1994; Khan, 1996; and Khan, 1998).

Nicotinic receptors are expressed in cellulodendritic domains as well as terminal domains of adrenergic neurons in the locus ceruleus, areas A5 and A7, serotonergic neurons in the nucleus raphe magnus and in cholinergic neurons (FIG. 15) (Iwamoto, 1993; Li, 1998; Mitchell, 1993; and Reuben, 2000). Thus, nicotine could activate adrenergic or serotonergic systems either through cellular action in the brain or by increasing transmitter release by acting at the axonal terminals in the spinal cord. Norepinephrine and serotonin have largely inhibitory actions at dorsal horn neurons (Garraway, 2001). However, activation of spinal α1-adrenergic receptors can have hyperalgesic actions (North, 1984). Similarly, nicotine can facilitate the release of acetylcholine that can have either an inhibitory or excitatory effect on dorsal horn cells through actions on muscarinic receptors (Garraway, 2001) (FIG. 15).

Potential Mechanisms for Volatile Isoflurane Hyperalgesia

1. Adrenergic Inhibition

Pontine noradrenergic neurons in the locus ceruleus and areas A5 and A7 have 2 major projections that are important in pain modulation (FIG. 15). Fibers from the pontine and medullary noradrenergic nuclei contribute to a pathway that modulates the activity of spinothalamic neurons in the dorsal horn. Noradrenergic modulation of spinal nociceptive transmission can be both facilitatory and inhibitory, but under most circumstances inhibition is dominant (Fields, 1991). Noradrenergic neurons predominantly from area A5 project to the serotonergic neurons in the nucleus raphe magnus (Sagen, 1986). This noradrenergic projection is largely inhibitory as electrolytic lesions and adrenergic antagonists are analgesic because of relief of inhibition of the serotonergic neurons in the nucleus raphe magnus.

It has been suggested that adrenergic projections from the brain to the spinal cord are required for the hyperalgesic response to isoflurane (Kingery, 2002). As described above, adrenergic projections from the brain cause modulation of nociceptive responses. Kingery and colleagues found that destruction of adrenergic neurons with ICV injection of the targeted adrenergic immunotoxin DβH-saporin reduced the analgesic effects of isoflurane (Kingery, 2002).

Kingery and colleagues found that six to seven days after spinal cord transection at the T7-8 level, animals no longer had a hyperalgesic effect from isoflurane, but analgesia was intact. They interpreted these data to indicate that hyperalgesia was due to interaction with isoflurane in the brain. An alternative interpretation is that six to seven days after spinal cord transection, the adrenergic (and serotonergic) axons likely retract after separation from the cell body. Another possible interpretation of these data is that the adrenergic (or serotonergic) axons are required for isoflurane hyperalgesia and the action of nicotine.

2. Serotonergic Inhibition

Serotonergic cells in the medullary nucleus raphe magnus and adjacent nucleus reticularis magnocellularis project to the spinal cord where they modulate nociceptive transmission. Serotonergic activity is largely inhibitory via activation of 5HT₁a and b receptors, but in the chronic pain setting can be facilitatory via activation of 5HT₃ receptors (Oyama, 1996). Approximately 20% of the neurons in the rostral ventral medulla are serotonergic (Fields, 1999). Most of the serotonergic neurons are termed neutral-cells, as opposed to “on” or “off” cells of the rostral ventral medulla (Potrebic, 1994; and Gao, 2000). These neurons are the exclusive source of serotonin in the dorsal horn, are tonically active and may modulate the activity of other descending systems (Gao, 1998).

Although activation of the serotonergic system is thought to in part, mediate nicotinic analgesia (Iwamoto, 1993; Rao, 1996; Hunt, 1998; Rueter, 2000; and Bitner, 1998), the role that the serotonergic system may play in isoflurane hyperalgesia is unknown. Isoflurane may induce a hyperalgesic state by inhibiting tonically active nicotinic acetylcholine receptors on the axonal terminals of serotonergic fibers thus decreasing the release of serotonin in the dorsal horn of the spinal cord. Studies on serotonin release in the spinal cord provide evidence for a tonically active nicotinic acetylcholine receptor controlling the release of serotonin (Cordero-Erausquin, 2001). Given the data supporting the involvement of the α-adrenergic system, serotonergic involvement would likely be parallel to adrenergic effects.

3. Muscarinic Inhibition

Nicotine treatment also leads to an increased release of acetylcholine in the spinal cord (Smith, 1989). Analgesia is a result of activation of postsynaptic muscarinic acetylcholine receptors (Chiari, 1999, and Smith, 1989).

4. Gender Differences in Isoflurane Hyperalgesia

Gender differences in pain responses have been widely reported. Epidemiological studies consistently reveal that women report more frequent and severe pain then men (Berkley, 1997; and Unruh, 1996). Pharmacodynamic differences in the drugs that are used to treat pain exist between the genders as well. For example, μ-opioid agonists are more effective in men while κ-opioid agents are more effective for postoperative pain in females (Cicero, 1996; Gear, 1999; Gear, 1996a; and Gear, 1996b) (reviewed in Berkley, 1997). In fact, women with postoperative pain had analgesia from the κ-opioid agonist nalbuphine, while men had hyperalgesia.

Gender is important in analgesia from nicotinic agonists also (Chiari, 1999; and Damaj, 2001). In both humans and rats, intrathecal administration of the acetylcholinesterase inhibitor, neostigmine, causes more analgesia in females than in males (Chiari, 1999). Neostigmine elevates acetylcholine concentration by reducing its degradation. As such it affects both muscarinic and nicotinic acetylcholine receptors. Chiari et al. found that while the muscarinic component was equal, female rats had a supplemental nicotinic component to the analgesia from neostigmine (Chiari, 1999). Female rats also had-more potent analgesia from RJR-2403, an α4β2 selective nicotinic agonist (Chiari, 1999). In contrast, in experiments with mice, females were less sensitive to analgesia from nicotine, administered subcutaneously or intrathecally (Damaj, 2001). Ovarian hormones were implicated in the difference in the potency of nicotine as treatment with both estrogen and progesterone reduced the analgesic effect of nicotine (Damaj, 2001). The difference in results may be attributable to the types of nicotinic receptors activated by the different agonists used, or a species difference between mice and rats.

Anesthesia in Clinical Practice

The hyperalgesic effect of volatile anesthetics appears to be a forgotten problem. When patients emerge from general anesthesia after surgery, they are expected to have pain and it is assumed that the pain is a result of surgical tissue damage. Although volatile anesthetics that are present on emergence have documented hyperalgesic effects, the etiology and neutralization of this effect for early postoperative pain has not yet been considered. In Dundee's studies, hyperalgesia was maintained for at least an hour (Dundee, 1960). While the duration of anesthesia prior to emergence was not documented, in these volunteer studies, it was not likely long. It is not known if hyperalgesia is maintained for a longer period of time after a longer anesthetic. After a longer anesthetic, there is a larger depot of anesthetic in less vessel rich tissues. As this anesthetic is removed from this reservoir at a rate proportional to blood flow, it may be excreted at low quantities for a prolonged period. This is thought to be the case with thiopental, which though it clears its anesthetic effect rapidly through redistribution, has a long terminal half-life and is excreted in low concentrations for over twenty-four hours.

The initial postoperative period is typically spent in a post-anesthesia recovery unit and is the most vulnerable period for respiratory and hemodynamic incidents. Postoperative pain is typically titrated during this period with opioid drugs that can have negative respiratory and hematological consequences. It is possible that the avoidance of hyperalgesic effects of anesthetic drugs could reduce early postoperative pain and provide a more stable recovery period.

### SUMMARY OF THE INVENTION

This invention provides a method for reducing hyperalgesia following a pain-inducing procedure being performed on a subject which comprises administering to the subject an anesthetic which is an antagonist of the subject's nicotinic acetylcholine receptor, in an amount effective to inhibit the subject's perception of pain during the pain-inducing procedure; then performing the pain-inducing procedure on the subject; and administering to the subject a hyperalgesia-reducing amount of an agonist of the subject's nicotinic acetylcholine receptor such that the subject's hyperalgesia following the procedure is reduced.

This invention further provides a method for reducing hyperalgesia following a pain-inducing procedure being performed on a subject which comprises administering to the subject an anesthetic which is an antagonist of the subject's nicotinic acetylcholine receptor, in an amount effective to inhibit the subject's perception of pain during the pain-inducing procedure; then performing the pain-inducing procedure on the subject; and administering to the subject a hyperalgesia-reducing amount of an agonist of the subject's nicotinic acetylcholine receptor and a pain-reducing amount of a narcotic agent such that the subject's hyperalgesia following the procedure is reduced.

This invention further provides a method for reducing hyperalgesia following a pain-inducing procedure being performed on a subject which comprises administering to the subject an anesthetic which is an antagonist of the subject's nicotinic acetylcholine receptor, in an amount effective to inhibit the subject's perception of pain during the pain-inducing procedure; then performing the pain-inducing procedure on the subject; and administering to the subject a hyperalgesia-reducing amount of an agonist of the subject's nicotinic acetylcholine receptor and a hyperalgesia-reducing amount of a steroid sex hormone such that the subject's hyperalgesia following the procedure is reduced.

This invention further provides an article of manufacture comprising a packaging material having therein a nicotinic acetylcholine receptor agonist and a label indicating a use of the agonist for reducing hyperalgesia in a subject having therein a hyperalgesia-inducing amount of an anesthetic that acts as a nicotinic acetylcholine receptor antagonist.

Finally, this invention provides an article of manufacture comprising a packaging material having therein a nicotinic acetylcholine receptor agonist and a label indicating a use of the agonist for reducing enhanced pain sensitivity in a subject following surgery on the subject, which surgery employs an anesthetic that acts as a nicotinic acetylcholine receptor antagonist.

### BRIEF DESCRIPTION OF THE FIGURES

FIG. 1

Isoflurane causes hyperalgesia in female mice. Isoflurane 0.28% maximally reduced HPWL in female mice by 29% from a baseline of 9.3±0.7sec. to 6.6+0.6 seconds (p<0.1,t-test; n=14). Increased HPWL occurred at concentrations above 0.86% that were associated with sedation. MAC in these animals was 1.5±0.1% and LORR (mean partial pressures bracketing postural response and lack of response to placing the animal in a supine position) occurred at 0.59 +0.2% isoflurane.

FIG. 2

Nicotinic antagonists mecamylamine and chlorisondamine cause hyperalgesia at some doses.

FIG. 3

Mecamylamine potentiates isoflurane hyperalgesia.

FIG. 4

Nicotine pretreatment prevents isoflurane hyperalgesia.

FIGS. 5A-5C

Interaction of isoflurane and mecamylamine on the activation of a4p2 nAChRs expressed in Xenopus oocytes.

FIG. 6

Effect of nicotine on the activation of α4β2 nAChRs expressed in Xenopus oocytes.

FIGS. 7A and 7B

A) cartoon representation of the hyperalgesic action of isoflurane is due to inhibition of heteromeric nAChRs by isoflurane. B) cartoon depiction of three pharmacologically distinct populations of nAChRs including a tonically activated presynaptic nAChRs.

FIG. 8

Effect of nicotinic genotype on isoflurane hyperalgesia. Hyperalgesia was reduced inβ2 nicotinic knockout mice (0.28% isoflurane, p<0.001).

FIG. 9

ICV nicotine does not prevent isoflurane hyperalgesia. While nicotine injected ICV causes analgesia either in air or in the presence of 0.38% isoflurane (p<0.01), there is no difference in the reduction in HPWL by isoflurane when the mice are treated with ICV nicotine (p>0.05, n=5).

FIG. 10

Isoflurane does not cause hyperalgesia in mice after norepinephrine depletion. In mice with depleted norepinephrine stores 14 days after treatment with DSP-4 (filled circles), isoflurane did not reduce HPWL at any concentration (p>0.05, t-test, n=10). Nicotine had no significant effect on HPWL in DSP-4 treated animals at baseline or in the presence of isoflurane (empty circles) (p>0.05, t-test, n=10).

FIG. 11

Treatment with atropine 5 mg/kg had no effect on the hyperalgesic response to isoflurane (p<0.01). Nicotine 1 mg/kg also remained protective against the hyperalgesia.

FIGS. 12A and 12B

Nicotinic facilitation of norepinephrine release in a spinal cord slice Prevention by isoflurane of tonic and nicotine facilitated release. Release of ³H-norepinephrine from a spinal cord slice (CPM over 2.5 minute/Total Uptake in slice; filled squares, n=6). a) Application of nicotine (1 mM) at 22 minutes via the perfusion buffer leads to facilitation of ³H-norepinephrine release (p<0.02). In the presence of 0.38% isoflurane (filled circles, n=4), there is no facilitated release and basal release is reduced. b) Isoflurane 0.38% reduces the tonic release of ³H-norepinephrine in the spinal cord slice.

FIG. 13

Male mice have little hyperalgesic response to isoflurane (filled squares) 2 weeks after castration (filled ovals), males have higher baselines and a significant hyperalgesic response to isoflurane.

FIG. 14

Variation of isoflurane hyperalgesia with the estrus cycle, and after oophorectomy. At stages of the estrus cycle when estrogen (stage 2, filled circles and 3, up-pointing triangles) or progesterone (stage 5, diamonds) are elevated, there is isoflurane has less hyperalgesic effect. Two weeks after oophorectomy (filled black squares), in the absence of ovarian steroids, isoflurane has a greater hyperalgesic effect at lower concentrations (t-test, p<0.01). In all cases, females had more isoflurane hyperalgesia than males (filled grey squares). Because this figure contains data from many experiments, the data have been normalized to baseline HPWL to simplify comparison. There were no significant differences in baseline HPWL at the different stages of the estrus cycle. After oophorectomy however, there was a significant increase in baseline HPWL (p<0.01, data not shown).

FIG. 15

Interaction of nicotinic receptors with descending adrenergic and serotonergic fibers thought to be involved in pain modulation.

FIG. 16

Visual Analog Scale (VAS) scores in post-operative patients treated with or without nicotine.

FIG. 17

Heart rate (beats per minute) in post-operative patients treated with or without nicotine.

FIG. 18

Diastolic and systolic blood pressure (mmHg) in post-operative patients treated with or without nicotine.

### DETAILED DESCRIPTION OF THE INVENTION

This invention provides a method for reducing hyperalgesia following a pain-inducing procedure being performed on a subject which comprises administering to the subject an anesthetic which is an antagonist of the subject's nicotinic acetylcholine receptor, in an amount effective to inhibit the subject's perception of pain during the pain-inducing procedure; then performing the pain-inducing procedure on the subject; and administering to the subject a hyperalgesia-reducing amount of an agonist of the subject's nicotinic acetylcholine receptor such that the subject's hyperalgesia following the procedure is reduced.

In one embodiment of the invention, performing the pain-inducing procedure on the subject and administering to the subject a hyperalgesia-reducing amount of an agonist of the subject's nicotinic acetylcholine receptor are performed simultaneously. In another embodiment of the invention, administering to the subject a hyperalgesia-reducing amount of an agonist of the subject's nicotinic acetylcholine receptor follows performing the pain-inducing procedure on the subject. In another embodiment of the invention, administering to the subject a hyperalgesia-reducing amount of an agonist of the subject's nicotinic acetylcholine receptor continues after the pain-inducing procedure on the subject is performed.

In a preferred embodiment of the invention, the subject is a human. In one embodiment of the invention, the subject is a male. In another embodiment of the invention, the subject is a female. In another embodiment of the invention, the female subject has a low level of circulating estrogen. In another embodiment of the invention, the female subject is post-menopausal.

In one specific embodiment of the invention, the hyperalgesia consists of an enhanced sensitivity to pain.

In another embodiment of the invention, the pain-inducing procedure is a surgical procedure.

In another embodiment of the invention, the anesthetic is isoflurane, halothane, sevoflurane, desflurane, nitrous oxide, ketamine or a barbiturate.

In a preferred embodiment of the invention the nicotinic acetylcholine receptor agonist is administered intranasally.

In another embodiment of the invention, the nicotinic acetylcholine receptor agonist is administered transdermally.

In one embodiment of the invention, the nicotinic acetylcholine receptor agonist is nicotine. In one embodiment the nicotine is administered intranasally. In a preferred embodiment of the invention, the nicotine dose is about 3 milligrams. In another preferred embodiment of the invention, the nicotine dose is about 3 milligrams and is administered intranasally.

In one embodiment of the invention, the nicotinic acetylcholine receptor agonist is administered via a single dose. In another embodiment of the invention, the nicotinic acetylcholine receptor agonist is administered via a plurality of doses.

In one embodiment of the invention, the nicotinic acetylcholine receptor agonist is administered while the subject is conscious. In another embodiment of the invention, the nicotinic acetylcholine receptor agonist is administered while the subject is unconscious.

This invention further provides a method for reducing hyperalgesia following a pain-inducing procedure being performed on a subject which comprises administering to the subject an anesthetic which is an antagonist of the subject's nicotinic acetylcholine receptor, in an amount effective to inhibit the subject's perception of pain during the pain-inducing procedure; then performing the pain-inducing procedure on the subject; and administering to the subject a hyperalgesia-reducing amount of an agonist of the subject's nicotinic acetylcholine receptor and a pain-reducing amount of a narcotic agent such that the subject's hyperalgesia following the procedure is reduced. In one embodiment of the invention, performing the pain-inducing procedure on the subject and administering to the subject a hyperalgesia-reducing amount of an agonist of the subject's nicotinic acetylcholine receptor and a pain-reducing amount of a narcotic agent are performed simultaneously. In another embodiment of the invention, administering to the subject a hyperalgesia-reducing amount of an agonist of the subject's nicotinic acetylcholine receptor and a pain-reducing amount of a narcotic agent follows performing the pain-inducing procedure on the subject. In another embodiment of the invention, administering to the subject a hyperalgesia-reducing amount of an agonist of the subject's nicotinic acetylcholine receptor and a pain-reducing amount of a narcotic agent continues. after the pain-inducing procedure on the subject is performed. In another embodiment, the narcotic agent is dilaudid, morphine, Demerol or fentanyl.

This invention further provides a method for reducing hyperalgesia following a pain-inducing procedure being performed on a subject which comprises administering to the subject an anesthetic which is an antagonist, of the subject's nicotinic acetylcholine receptor, in an amount effective to inhibit the subject's perception of pain during the pain-inducing procedure; then performing the pain-inducing procedure on the subject; and administering to the subject a hyperalgesia-reducing amount of an agonist of the subject's nicotinic acetylcholine receptor and a hyperalgesia-reducing amount of a steroid sex hormone such that the subject's hyperalgesia following the procedure is reduced. In one embodiment of the invention, performing the pain-inducing procedure on the subject and administering to the subject a hyperalgesia-reducing amount of an agonist of the subject's nicotinic acetylcholine receptor and a hyperalgesia-reducing amount of steroid sex hormone are performed simultaneously. In another embodiment of the invention, administering to the subject a hyperalgesia-reducing amount of an agonist of the subject's nicotinic acetylcholine receptor and a hyperalgesia-reducing amount of steroid sex hormone follows performing the pain-inducing procedure on the subject. In another embodiment of the invention, administering to the subject a hyperalgesia-reducing amount of an agonist of the subject's nicotinic acetylcholine receptor and a hyperalgesia-reducing amount of steroid sex hormone continues after the pain-inducing procedure on the subject is performed.

In one specific embodiment of the invention, the sex steroid hormone is a synthetic sex steroid hormone. In another embodiment of the invention, the sex steroid hormone is estrogen. In another embodiment of the invention, the sex steroid hormone is a synthetic estrogen. In another embodiment of the invention, the sex steroid hormone is testosterone. In another embodiment of the invention, the sex steroid hormone is a synthetic testosterone. In another embodiment of the invention, the sex steroid hormone is progesterone. In another embodiment of the invention, the sex steroid hormone is a synthetic progesterone.

In one specific embodiment of the invention, the nicotinic acetylcholine receptor agonist is nicotine. In a preferred embodiment of the invention, the nicotinic acetylcholine receptor agonist is administered intranasally. In another embodiment of the invention, the nicotinic acetylcholine receptor agonist is administered transdermally.

In one specific embodiment of the invention, the nicotinic acetylcholine receptor agonist is administered via a single dose. In another embodiment of the invention, the nicotinic acetylcholine receptor agonist is administered via a plurality of doses. In a further embodiment of the invention, the nicotinic acetylcholine receptor agonist is administered while the subject is conscious. In another embodiment of the invention, the, nicotinic acetylcholine receptor agonist is administered while the subject is unconscious.

This invention further provides an article of manufacture comprising a packaging material having therein a nicotinic acetylcholine receptor agonist and a label indicating a use of the agonist for reducing hyperalgesia in a subject having therein a hyperalgesia-inducing amount of an anesthetic that acts as a nicotinic acetylcholine receptor antagonist. In the preferred embodiment of the invention, the nicotinic acetylcholine receptor agonist is nicotine.

This invention further provides an article of manufacture comprising a packaging material having therein a nicotinic acetylcholine receptor agonist and a label indicating a use of the agonist for reducing enhanced pain sensitivity in a subject following surgery on the subject, which surgery employs an anesthetic that acts as a nicotinic acetylcholine receptor antagonist. In the preferred embodiment of the invention, the nicotinic acetylcholine receptor agonist is nicotine.

As used herein, an “agonist” of a receptor is an agent that interacts with the receptor so as to elicit a biological response which is of the same kind as the biological response elicited by the receptor's natural ligand. An agonist can be, for example, naturally occurring or synthetic. A “partial agonist” of a receptor is an agent that interacts with the receptor so as to elicit a biological response which (i) is of the same kind as the biological response elicited by the receptor's natural ligand, but (ii) regardless of dosage, is of a smaller magnitude than the maximum biological response elicited by the receptor's natural ligand.

This invention further provides a method for reducing hyperalgesia in a subject having therein a hyperalgesia-inducing amount of an anesthetic which acts as a nicotinic acetylcholine receptor antagonist, comprising administering to the subject a hyperalgesia-reducing amount of a nicotinic acetylcholine receptor agonist, thereby reducing hyperalgesia in the subject. As used herein, the term “subject” shall mean any animal including, without limitation, a human, a mouse, a rat, a rabbit, a non-human primate, or any other mammal. In the preferred embodiment, the subject is human. The subject can be male or female. In one additional embodiment, the subject is a female who has a low level of circulating estrogen, such as female who is postmenopausal.

In one specific embodiment, the hyperalgesia consists of an enhanced sensitivity to pain. “Enhanced pain sensitivity” and “enhanced sensitivity to pain” are used synonymously herein, and shall mean a sensitivity to pain which is greater than the sensitivity to pain which occurs in a subject in the absence of any anesthetic which is a nicotinic acetylcholine receptor antagonist.

The anesthetic that causes hyperalgesia can be any such anesthetic known in the art, such as isoflurane, halothane, sevoflurane, desflurane, nitrous oxide, ketamine or a barbituate. Likewise, the nicotinic acetylcholine receptor agonist can be any such agonist known, such as nicotine or derivatives thereof, or other such agonists described above. The nicotinic acetylcholine receptors on which the agonist acts can comprise any permutation of α and β subunits as set forth above in Table 1, as well as any heterologous variant thereof (e.g. α₂α₃β₂β₃β₄).

In this invention, administering nicotinic acetylcholine receptor agonist can be effected or performed using any of the various methods and delivery systems known to those skilled in the art. The administering can be performed, for example, intravenously, orally, nasally, via implant, transmucosally, transdermally, intramuscularly, and subcutaneously. The following delivery systems, which employ a number of routinely used pharmaceutical carriers, are only representative of the many embodiments envisioned for administering the instant compositions.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

Determining an effective amount of nicotinic acetylcholine receptor agonist for use in the instant invention can be done based on animal data using routine computational methods. In one embodiment, the effective amount, administered intranasally, is between about 1 mg and about 5 mg of nicotinic acetylcholine receptor agonist (e.g. nicotine). In another embodiment, the effective amount, administered intranasally, is between about 0.5 mg and about 5 mg of nicotinic acetylcholine receptor agonist. In the preferred embodiment, the effective amount, administered intranasally, is about 3 mg of nicotinic acetylcholine receptor agonist. In another embodiment, the effective amount, administered transdermally, is a dosage determined based on dosages used in commercially available nicotine transdermal patches. In one embodiment of the instant method, the nicotinic acetylcholine receptor agonist is administered in a single dose. In another embodiment, the nicotinic acetylcholine receptor agonist is administered in multiple doses. The nicotinic acetylcholine receptor agonist can be administered to the subject while conscious or unconscious.

In a further embodiment, the nicotinic acetylcholine receptor agonist is administered to the subject in addition to a pain-reducing amount of a narcotic agent. The narcotic agent and the nicotinic acetylcholine receptor agonist can be administered together or separately. In another embodiment, the narcotic agent is morphine, Demerol or fentanyl.

In a further embodiment, the nicotinic acetylcholine receptor agonist is administered to the subject with an amount of estrogen, progesterone and/or testosterone effective to reduce hyperalgesia. The agonist can be administered together with, or separately from, the estrogen, progesterone and/or testosterone.

This invention also provides a method for reducing enhanced pain sensitivity in a subject following surgery on the subject, which surgery employs an anesthetic that acts as a nicotinic acetylcholine receptor antagonist, comprising administering to the subject at a suitable time following the surgery, an amount of a nicotinic acetylcholine receptor agonist effective to reduce enhanced pain sensitivity, thereby reducing enhanced pain sensitivity in the subject.

In one embodiment of this method, the nicotinic acetylcholine receptor agonist can be administered to the subject while conscious or unconscious.

This invention also provides an article of manufacture comprising a packaging material having therein a nicotinic acetylcholine receptor agonist and a label indicating a use of the agonist for reducing hyperalgesia in a subject having a hyperalgesia-inducing amount of an anesthetic that acts as a nicotinic acetylcholine receptor antagonist. In the preferred embodiment, the nicotinic acetylcholine receptor agonist is nicotine.

Finally, this invention provides an article of manufacture comprising a packaging material having therein a nicotinic acetylcholine receptor agonist and a label indicating a use of the agonist for reducing enhanced pain sensitivity in a subject following surgery on the subject, which surgery employs an anesthetic that acts as a nicotinic acetylcholine receptor antagonist. In preferred embodiment, the nicotinic acetylcholine receptor agonist is nicotine.

All embodiments of the instant method for reducing hyperalgesia are envisioned mutatis mutandis, as applicable, with respect to the instant method for reducing enhanced pain sensitivity and the instant articles of manufacture.

This instant invention is illustrated in the Experimental Details section that follows. This section is set forth to aid in an understanding of the instant invention but is not intended to, and should not be construed to, limit in any way the invention as set forth in the claims which follow thereafter.

Experimental Details

A. Synopsis

Volatile anesthetic at the low concentrations present on emergence from anesthesia increases sensitivity to pain. This enhanced pain sensitivity can last upwards of 1 hour in humans after general anesthesia. This invention provides a new method for postoperative pain treatment, based on the surprising discovery that nicotine ameliorates hyperalgesia in animals.

Mice are a useful animal model for anesthetic hyperalgesia that has been demonstrated previously in rats and humans (Dundee, 1960; Zhang, 2000; Briggs, 1982; Ewen, 1995; Archer, 1996; Tatsuo, 1997; and Tatsuo, 1999). Genetically altered mice are available and to help elucidate important molecular components in the mechanism of anesthetic induced hyperalgesia.

Experiments demonstrating aspects of this invention have focused on isoflurane as an anesthetic because it is a volatile anesthetic that is commonly used in humans. However, all volatile anesthetics that have been tested cause hyperalgesia (Zhang, 2000) and the application of this invention applies to all anesthetic agents that are nAChRs antagonists.

B. Methods

HPWL Measurement

With approval of the UCSF Committee on Animal Research, we studied female, 129J strain mice at 6-8 weeks of age, weighing 15-20 grams obtained from the Jackson Laboratories (Bar Harbor, Maine). Hind paw withdrawal latency (HPWL) was measured with a modification of the automatic device (Plantar Tes, Ugo Basile Biological Research Apparatus, Comerio, Italy) described by Hargreaves et al. (Hargreaves, 1987) in up to five unrestrained mice (per study) housed individually in clear plastic chambers. The chambers rested on a clear glass plate. Over the chambers a clear Plexiglas enclosure was placed so that it rested on a silicone rubber gasket that produced a seal to the glass plate. Gas tight fittings at either end permitted delivery to and scavenging of isoflurane. Isoflurane in oxygen was delivered from a variable-bypass vaporizer. Concentrations of isoflurane were monitored with an infrared analyzer (RGM, Datex-Ohmeda, Madison, Wis.) and analyzed at the end of each concentration step with gas chromatography. The chromatograph reading was accepted as the value for the exposure concentration. Heating strips warmed the glass plate to minimize body heat loss. To diminish exploratory activity, the mice were acclimated to this environment for at least thirty minutes before commencing the study. After acclimation, a movable source of radiant heat was applied from a projector lamp (Radium tungsten halogen lamp, model EJY, 19V, 80 W; General Electric, Glen Allen, Va.) through a 7 mm aperture under the glass plate to the hind paw of the resting mouse. A photocell within the housing that surrounds the lamp sensed the light reflecting from the hind paw of the mouse (i.e., whether the paw remained in place). The device automatically measured the time from the onset of application of the light (heat) to the time the mouse moved the hind limb (as determined by the moment the light no longer reflected from the paw to the photocell).

Animals were allocated into four study groups: saline, mecamylamine (Sigma, Milwaukee, Wis.), chlorisondamine (Tocris, Ballwin, Mo.), or nicotine (Sigma, Milwaukee, Wis.) each drug administered by intraperitoneal injection. 5-28 mice were studied per group. Some mice were used for more than one study and at least two days separated such studies. In all experiments,, HPWL measurement was made for each hind paw five times (total of 10 measurements). Measurements on each paw were made at approximately five-minute intervals. The ten readings were averaged to produce the value for each control or anesthetic level. After obtaining control measurements, isoflurane (Abbot Laboratories, North Chicago, Ill.) was delivered in a stepwise manner at inspired concentrations of 0.14%, 0.28%, 0.56%, 0.84 and, in some cases, 0.98% inspired concentration of isoflurane (i.e., 0.1, 0.2, 0.4, 0.6 and 0.7 MAC; MAC for isoflurane equals 1.4% in these mice). At the end of equilibration, HPWL was determined. After the final equilibration, anesthetic delivery was discontinued, and after 1 hour HPWL was again measured to demonstrate recovery. All animals returned to control HPWL within 1 hour.

In all experiments with mecamylamine, chlorisondamine and their saline controls, animals were injected intraperitoneally (IP) with mecamylamine in a saline solution or saline (control) at a volume of 10 ml/kg, at least 30 minutes before HPWL testing. The duration of mecamylamine's action was tested with two control experiments. First, 5 mice were injected with mecamylamine 5 mg/kg IP or saline. These mice were then tested at 1-hour intervals with nicotine 1 mg/kg IP. The mice that were previously treated with mecamylamine did not show prostration from the nicotine for up to 4 hours, indicating continued blockade by mecamylamine during this time period. Untreated mice lay prone within 5 minutes. Second, in order to determine whether the analgesic effects of mecamylamine were stable over the testing period, 5 mice were tested immediately with 0.84% isoflurane which is normally the anesthetic concentration tested 3 hours after mecamylamine treatment. There was no significant difference in the response to 0.84% isoflurane whether the mice were tested at 1 or 3 hours after treatment with mecamylamine. Mecamylamine plasma concentration was measured in 5 female mice, 1 hour after I.P. injection using a combination of Gas Chromatography and Mass Spectroscopy described by Jacob et al. (Jacob, 2000).

Because nicotine's analgesic effect is known to be short lived (Damaj, 2001), mice were injected IP with (s)-(−)-nicotine (Sigma, Milwaukee, Wis.) 1 mg/kg or saline in a total volume of 10 ml/kg, 5 minutes before HPWL testing. In mice, nicotine at 1 mg/kg reaches a peak concentration of approximately 2 μM at 5 minutes and is undetectable by HPLC at 40 minutes (Thompson, 1982). In studies with nicotine, mice breathed oxygen or the desired anesthetic concentration for 25 minutes, were injected with nicotine or saline, and then were re-equilibrated for 5 minutes prior to HPWL testing. Injections of nicotine were separated by at least 1 hour. As each testing period lasted approximately 25 minutes, the peak effects of nicotine were determined with this methodology. The first HPWL measurements for each paw were not significantly different than the last HPWL measurements in these experiments. The control animals studied with the multiple injection protocol had a slightly different baseline than control animals that did not receive multiple injections, thus animals tested with nicotine are compared to their own controls.

Electrophysiology

The human α4 and β2 type nAChRs were in a pSP64 expression vector. Standard techniques were used to linearize the vectors and use them as templates to make cRNA using SP6 as the polymerase. The human nicotinic clones were a gift from Dr. Jon Lindstrom, Ph.D. (Department of Neuroscience, University of Pennsylvania, Philadelphia, Pa.).

Xenopus laevis oocytes were removed from the females and defolliculated with collagenase. After the oocytes rested for 24 hours in L-15 oocyte medium, about 10 ng of a 1:1 ratio of α4 to β2 cRNA were injected into individual oocytes. A manual injector was used for this process (Nanoject; Drummond Scientific, Broomall, Pa.). The oocytes were incubated for 2-5 days in ND-96 medium (NaCl 96 mM, KCl 2 mM, MgCl₂ 1 mM, CaCl₂ H₂O 1.8 mM, HEPES 5 mM, Na-pyruvate 2.5 mM, theophylline 0.5 mM, and 10mg/L of gentamicin, adjusted to pH 7.5).

Whole oocytes were used to record currents using a Gene-Clamp 500 two-microelectrode voltage-clamp amplifier with an active ground (Axon Instruments, Inc., Foster, Cailf.). The recording electrodes were pulled from glass capillary tubing (Drummond, Broomall, Pa.) to obtain a resistance between 1 and 5 MΩ and filled with 3M KCl. Ba² Ringer's solution was used as the extracellular solution in order to avoid current amplification by calcium activated chloride currents (115 mM NaCl, 2.5 mM KCl, 1.8 mM BaCl₂, 10 mM HEPES, 1 μM atropine, pH 7.4). Atropine was included to avoid activation of intrinsic muscarinic receptors. Experiments were performed at room temperature. Isoflurane was prepared from a saturated solution by serial dilution. Concentrations were verified by gas chromatography.

Oocytes were tested at a membrane potential of −60 mV. Bolus application of the agonist +/− indicated antagonist(s) was applied at a rate of 4 ml/min for a 2 second application.

Antagonists were pre-applied for 2 minutes prior to activation. Concentration response curves were made from the percent change in peak current from ACh activation in the presence of antagonist(s), compared to ACh alone. As the ACh concentration at central neuronal nAChRs is unknown, 1 mM ACh (saturating) was used to detect inhibition by isoflurane and mecamylamine experiments with mecamylamine. ACh 2 μM was used to detect potentiation by nicotine. Currents were measured in 5-8 cells for each data point. Clampex 7 (Axon instruments, Foster City, Calif.) was used for data acquisition.

Statistical Analysis

Microcal Origins 5.0 (Microcal, Northampton, Mass.) was used for statistical calculations and graphical presentation. The in vitro data were fit to a modified Hill equation, y=100/(1+(x/IC₅₀)ⁿ), where IC₅₀ is the concentration of drug at which of 50% of the response is inhibited and n is the Hill coefficient. Interaction between isoflurane and mecamylamine was interpreted using an isobolographic analysis in which the concentrations that cause 50% inhibition of α4β2 nAChR activation are displayed graphically with a line of additivity and 95% confidence intervals. The concentrations of the combined drugs that cause 50% inhibition are displayed, those that fall within the 95% confidence intervals are considered to interact additively (Tallarida et al., 1989).

HPWL data for females in response to isoflurane and mecamylamine had a biphasic response, thus the extent of maximal hyperalgesia in the presence of isoflurane was compared to baseline with a paired t-test, using the Bonferroni correction for multiple comparisons.

Oophorectomy

Under isoflurane anesthesia, a 3 mM incision was made caudal to the inferior pole of the kidney. The ovary and oviduct was tied with a double 3.0 polysorb suture. The fascia was closed with 3.0 polysorb and the skin with 4.0 nylon. The procedure was repeated on the other side. The mouse was injected with buprenophine 1 mg/kg prior to emergence for pain control.

Spinal Cord Slice Superfusion

Spinal Cord Slice Preparation: The spinal cord was dissected from the mouse. Under CO₂ narcosis the mouse was decapitated. The spinal column was removed and placed into cold dissection solution where the spinal cord was removed with a dissection microscope. The lumbar section of the cord was isolated and imbedded in a 3% low melting point agar (Fisher Scientific) block made in dissection solution. Transverse 400 micrometer slices were made using a Vibratone VT1000S slicing apparatus (Leica, Whetzlar, Germany). Slicing was performed in ice cold Krebs solution bubbled with 5%CO₂/95%O₂.

The cord slices were incubated in a solution of 1 ml of Krebs solution and 10 ul of 1-[7,8-³H] noradrenaline in a closed environment surrounded by Krebs solution bubbled with CO₂/O₂ for 30 minutes. The slices were transferred into a superfusion and stimulation chamber (Warner Instruments Inc.,) where Krebs solution was allowed to perfuse over them for 30 minutes at a rate of 0.5 ml/min. The superfusion chamber thermostatically temperature controlled at 37° C. The solutions were pumped to the chamber at a constant rate of 0.5 ml/min using a Kwik-Pump 290 (Long). Following a 30 minute period for equilibration, collections were taken every 1-2.5 minutes. Ecolite scintillation fluid (15 ml) was then added to each collection vial. The samples were analyzed for radioactivity (CPM) using a Packard TRI CARB 2100 TR scintillation counter (Meriden, Conn.).

Spinal Cord Slice Solution

Dissection Krebs Solution (mM) NaCl 125, KCl 2.5, NaHCO₃ 26, NaH₂PO₄-H₂O_{1.25}, MgCl₂ 6,CaCl₂ 1.5, glucose 2.5. Perfusion Krebs Solution (mM) NaCl 125, KCl 2.5, NaHCO₃ 26, NaH₂PO₄—H₂O 1.25, MgCl₂ 1,CaCl₂ 2, glucose 2.5.

Drugs

All chemicals and salts were from Sigma/Aldrich(St. Louis Mo.) except: Atropine Sulfate (ICN; Costa Mesa, Calif.); L-[7,8-³H]-Noradrenaline (1.33 TBq/mmol) (Amersham Scientific; Piscataway, N.J.); and Chlorisondamine (Tocris; Ellisville, Mo.).

C. Results and Discussion

Experimental Set I

Isoflurane Hyperalgesia in Female Mice

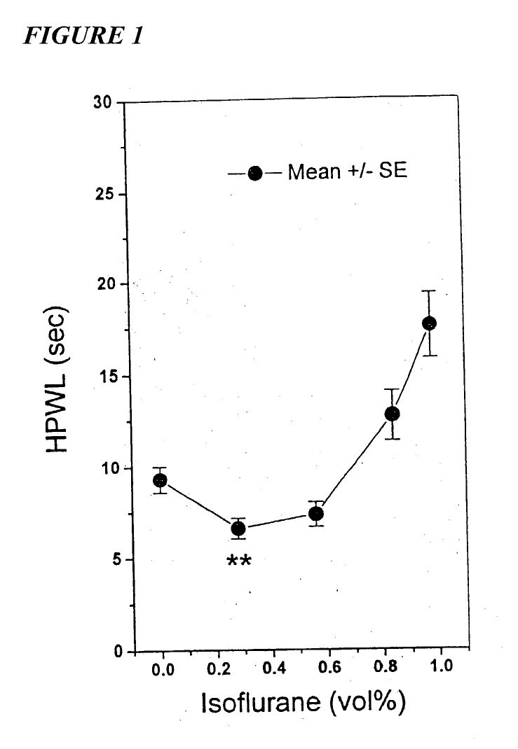

The nociceptive response produced by 0.28 to 0.98% isoflurane was tested by measuring HPWL in female mice (FIG. 1). The mice were significantly hyperalgesic while breathing 0.28% isoflurane as compared to the oxygen control (FIG. 1) (t-test, p<0.01). HPWL returned to baseline at 0.56% isoflurane, and higher isoflurane concentrations resulted in progressively increasing analgesia. HPWL returned to baseline by 1 hour after isoflurane washout in all mice.

Behavioral Effects of Nicotinic Antagonists

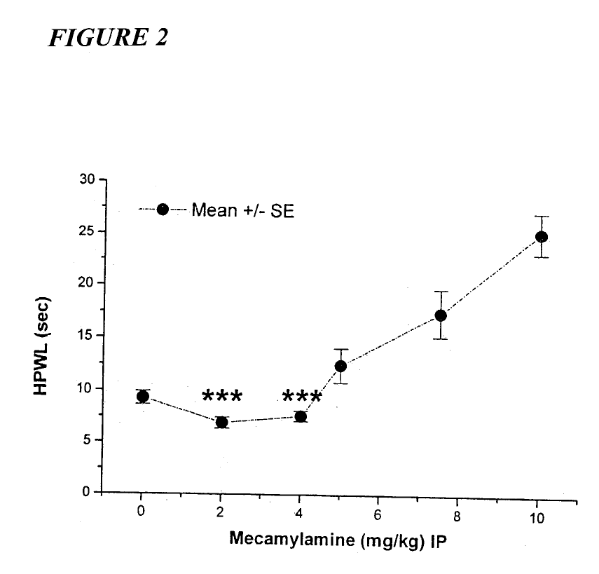

Mecamylamine intraperitoneally administered to female mice caused a biphasic response, with significantly increased nociception at 2 and 4 mg/kg IP (t-test; p<0.001), and analgesia at doses of 5 mg/kg and greater (FIG. 2). Mice assumed a hunched posture, made rapid back and forth rocking motions, and aggressively groomed themselves after injection of mecamylamine at 7.5 or 10 mg/kg. The plasma concentration of mecamylamine was measured with GC-Mass spectroscopy, 1 hour after I.P. injection of 5 mg/kg in female mice and was found to be 203+/−67 nM.

Effects of Nicotinic Antagonists on Isoflurane Induced Hyperalgesia

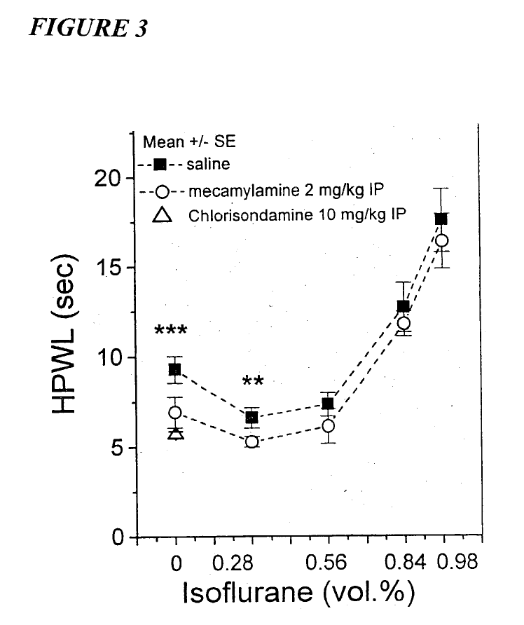

The hyperalgesia induced by 0.28% isoflurane in female mice was enhanced by mecamylamine 2 mg/kg (t-test p<0.01)(FIG. 3). At higher concentrations of isoflurane that caused analgesia, HPWL was not changed by mecamylamine. Mecamylamine 5 mg/kg caused hyperalgesia at baseline (FIG. 2), but the addition of isoflurane 0.28% caused a 50% decrease in HPWL. Chlorisondamine 10 mg/kg, a nicotinic antagonist, also caused hyperalgesia (FIG. 3).

Effect of Nicotine of Isoflurane Hyperalgesia

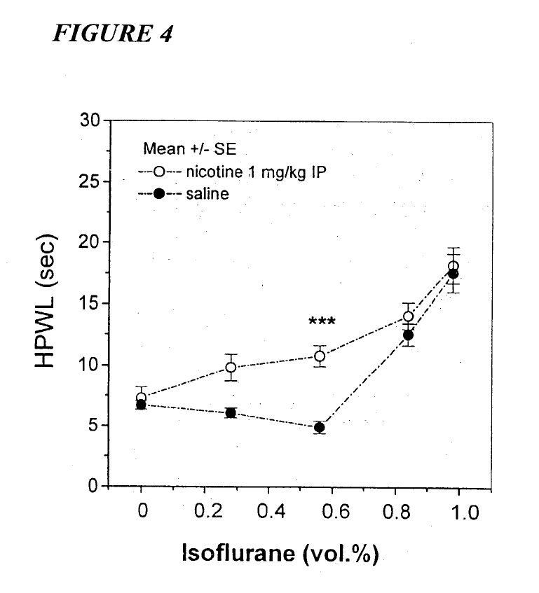

Nicotine can produce analgesia at high concentrations when given systemically, IT and ICV (Damaj, 2001; Mattila, 1968; and Lloyd, 1998). The -prototypical nicotinic agonist, nicotine was tested to see if it might reverse the hyperalgesic response caused by isoflurane. A concentration of nicotine was chosen that when it is given systemically it is not analgesic (1 mg/kg IP). Although 1mg/kg IP nicotine did not cause significant analgesia in female mice at baseline (FIG. 4), it prevented the hyperalgesic properties of isoflurane, with maximal effect at 0.56% isoflurane (t-test, p<0.001). The action of nicotine to prevent isoflurane hyperalgesia was specific for the hyperalgesic phase, as it had no effect at baseline or at concentrations of isoflurane (>0.58%) that produced analgesia in female mice.

Because of the short half-life of nicotine, in these experiments (FIG. 4), animals received an injection of either nicotine or saline 5 minutes prior to each testing period. The HPWL responses to isoflurane in the saline injected animals differ using this paradigm in that baseline HPWL is lower and maximal hyperalgesia is achieved with 0.56% isoflurane instead of 0.28% isoflurane.

Interaction of Isoflurane and Mecamylamine on the Activation of α4β2 nAChRs Expressed in Xenopus Oocytes

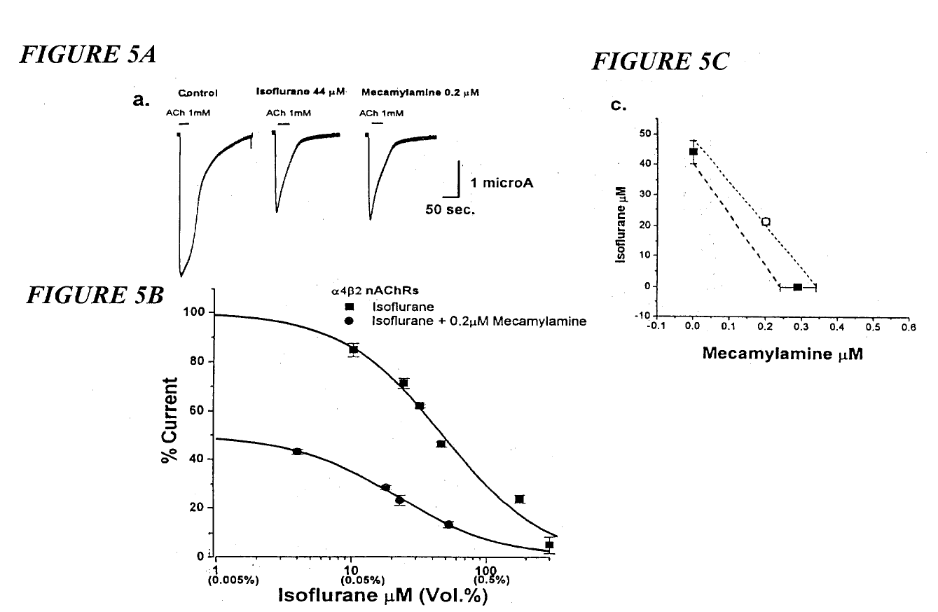

Isoflurane caused hyperalgesia in female mice within the same low concentration range (0.28-0.56% or 63-128 μM) as α4β2 nAChRs were inhibited in vivo (Flood, 1997; Violet, 1997; and Cardoso, 1999) (FIG. 5). In order to provide additional evidence for a role for the nAChR in the nociceptive response to isoflurane, effects of nicotine and mecamylamine on isoflurane inhibition of α4β2 nAChRs were studied at concentrations relevant to those used in the behavioral experiments.

Both isoflurane and mecamylamine act as non-competitive antagonists at heteromeric nAChRs (FIGS. 5A and 5B) (Violet, 1997; and Webster, 1999). In order to study the role of nicotinic modulation in the isoflurane nociceptive response, we evaluated the interaction between isoflurane and mecamylamine in vitro on heteromeric nAChRs. FIG. 5A shows representative current traces from α4β2 nAChRs activated by 1 mM ACh alone, in the presence of 44 μM isoflurane or 0.2 μM mecamylamine. The half maximal inhibitory concentration for isoflurane inhibition of α4β2 nAChRs was 44 μM. The concentration of mecamylamine used was approximately IC₅₀ for inhibition of the α4β2 nAChR (0.29±0.05 μM) and was close to the mecamylamine concentration measured in plasma from female mice injected with 5 mg/kg (0.20±0.07 μM). A concentration response relationship for inhibition of α4β2 nAChRs by isoflurane with and without mecamylamine 0.2 μM is shown in FIG. 5B. Isobolographic analysis in FIG. 5C indicates that inhibition of α4β2 nAChR activation by mecamylamine and isoflurane applied together is within the 95% confidence intervals for additivity.

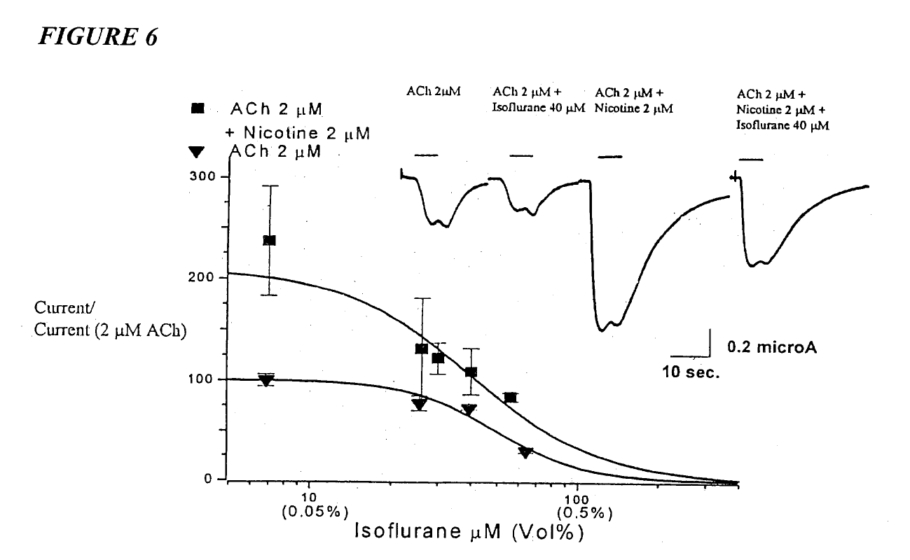

As expected, the addition of nicotine 2 μM (the approximate concentration measured by HPLC in a mouse injected with nicotine 1 mg/kg I.P.) (Thompson, 1982) to ACh 2 μM, produces a larger current than ACh alone (FIG. 6). In the presence of a given concentration of isoflurane, currents generated with ACh 2 μM nicotine 2 μM are always larger than those generated by ACh 2 μM alone.

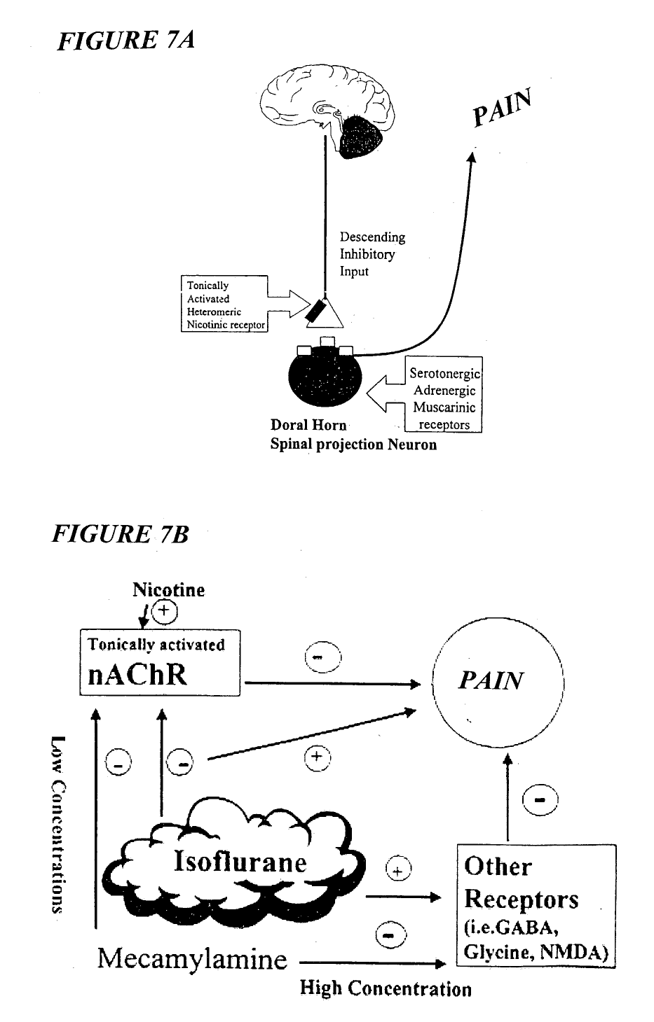

Several lines of evidence, summarized in cartoon form in FIG. 7A, suggest that the hyperalgesia action of isoflurane is due to the inhibition of heteromeric nicotinic acetylcholine receptors by isoflurane, while the analgesic phase is mediated by another mechanism.

1) Isoflurane inhibits the activation of the most common nicotinic subunit combination expressed in the CNS within the same concentration range in which hyperalgesia occurs in vivo (FIGS. 1 and 4). Although our in vitro experiments were conducted on nAChRs of human origin, little difference in the effect of isoflurane between species as diverse as chick, rat and human has been identified (Flood, 1997; Violet, 1997; and Cardoso, 1999).

2) Mecamylamine and isoflurane, both non-competitive nicotinic inhibitors, cause a similar biphasic nociceptive response in the female, with hyperalgesia at low concentrations that are more specific for nicotinic inhibition (O'Dell, 1988; McDonough, 1995; and Flood, 1998). Mecamylamine potentiates the hyperalgesia caused by isoflurane (FIG. 3). Chlorisondamine 10 mg/kg, another nicotinic antagonist also causes hyperalgesia (FIG. 3) presumably through inhibition of tonic nicotinic activity.

3) Nicotine, an agonist, specifically prevents isoflurane hyperalgesia in females at a concentration that does not cause analgesia alone or effect analgesic concentrations of isoflurane (FIG. 4).

Taken together, these findings suggest that nicotinic blockade mediates isoflurane's hyperalgesic effect, while other mechanisms may contribute to isoflurane's analgesic actions. It is unlikely that isoflurane analgesia is caused by heteromeric nicotinic inhibition as it is unaffected by nicotine and mecamylamine. At high concentrations, both isoflurane and mecamylamine are known to have activity other than nicotinic targets. Isoflurane modulates the activation of receptors for GABA (Lin, 1992; Harrison, 1993; and Hall, 1994) glycine (Harrison, 1993; and Downie, 1996) and glutamate (Carla, 1992; Dildy-Mayfield, 1996; and Minami K, 1998) at concentrations higher than those relevant for nAChR inhibition. Mecamylamine has NMDA antagonist properties at high concentrations in the 100 μM range (O'Dell, 1988; and Papke, 2001). Mecamylamine inhibits seizures induced by NMDA with an ED₅₀ of 12±3.2 mg/kg (McDonough, 1995). The analgesic properties of high concentrations of isoflurane and mecamylamine are more likely to be mediated through one or more of the above or other mechanisms.

The analgesic activity of nicotinic agonists is mediated, in part through modulation of the descending 5HT₃ projections from the raphe magnus (Bitner, 1998; and Rueter, 2000). Cordero-Erausquin et al. have recently proposed a model for nicotinic modulation of 5HT₃ transmission based on pharmacological modulation of 5HT₃ release in the mouse spinal cord (Cordero-Erausquin, 2001). They propose the existence of three pharmacologically distinct populations of nAChRs including a tonically activated presynaptic nAChR represented in FIG. 7B. While the experiments described above were not designed to differentiate between a brain and spinal action of isoflurane or to detect interaction with other neurotransmitters, their model may suggest one potential mechanistic explanation for our findings. Inhibition of a tonically activated excitatory receptor by isoflurane or mecamylamine at low concentration would be expected to reduce the release of serotonin and on this basis cause hyperalgesia. The analgesic properties of systemically-administered nAChR agents are also mediated by descending noradrenergic and muscarinic inhibitory pathways in addition to serotinergic pathways and the involvement of these systems cannot be ruled out (Rueter, 2000).

The nicotinic analgesic system is particularly important, and tonically active in the female. All volatile anesthetics tested by Zhang et al. produced hyperalgesia at low concentrations (Zhang, 2000). The concentrations of volatile anesthetics that cause hyperalgesia in animals (0.1-0.38% isoflurane) are commonly present in patients on emergence from general anesthesia. The significant incidence of emergence agitation when volatile anesthetics are used may be in part due to hyperalgesia from residual anesthetic.

Experimental Set II

Subunit Composition of the Nicotinic Acetylcholine Modulating Isoflurane Hyperalgesia

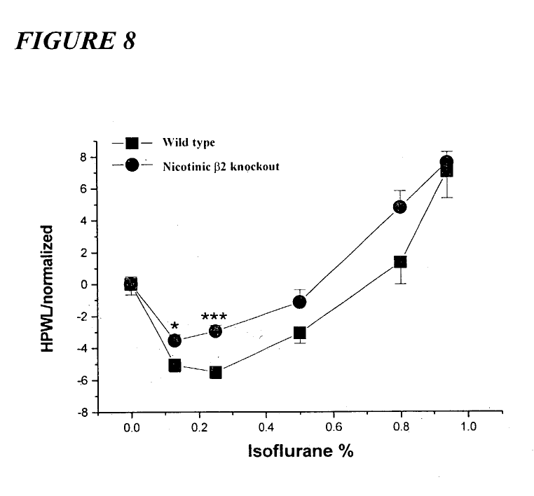

The subunit composition of the nicotinic receptors responsible for the analgesic effects of nicotinic agonists is controversial. Studies of mice lacking α4 and or β2 nicotinic subunits and some pharmacological studies suggest that receptors containing both subunits αre required for the analgesic effects of nicotine (Marubio, 1999; and Damaj, 1998). Nicotinic antagonists specific for α7 containing nicotinic achetylcholine receptors can be analgesic in some settings (Damaj, 2000). However, some studies with nicotinic antagonists suggest that a nicotinic acetylcholine receptor not composed of α4, β2 or α7 subunits is responsible for nicotinic analgesia (Reuter, 2000). Logic would suggest that several nicotinic receptors composed of different subunits play roles in analgesia under different conditions (i.e. spinal vs. ICV nicotine). The importance of the nicotinic β2 subunit in male C57/B16 mice and their generation matched wild type controls have been tested (a gift from J. P. Changeux, Institute Pasteur). The hyperalgesic effect of isoflurane is clearly reduced in these animals, but not absent (FIG. 8).

Nicotine Action on the Brain or Spinal Cord Preventing Isoflurane Hyperalgesia

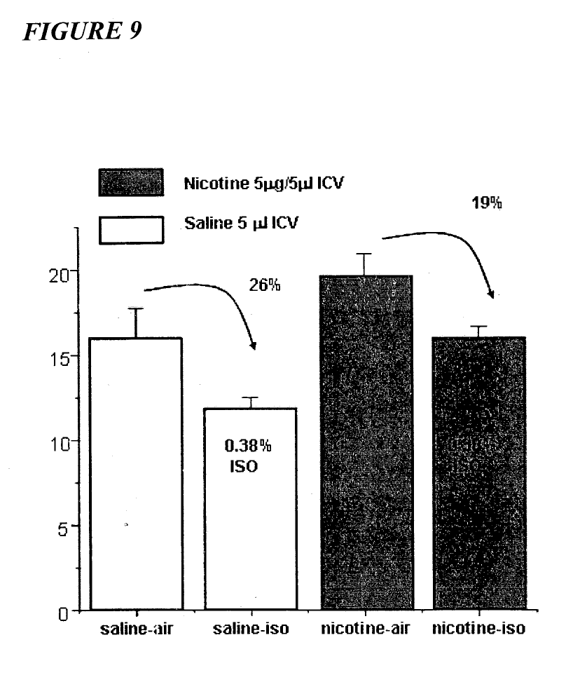

Nicotinic acetylcholine receptors are involved in the mechanism of isoflurane hyperalgesia, and nicotinic inhibition could be the initiating event. Since nicotine easily crosses the blood brain barrier, the systemic nicotine treatment could have been acting on receptors in the brain, spinal cord or less likely, in the periphery. Although peripheral sensory afferents express nicotinic receptors, the peripheral nicotinic antagonist hexamethonium does not prevent nicotinic analgesia. Since nicotine's analgesic action is likely due to actions on central nicotinic acetylcholine receptors, mice were given either nicotine (5 μg in 5 μl ) or an equal volume of saline by ICV injection (as described by Pedigo et al.) while the animals breathed oxygen or 0.38% isoflurane (the isoflurane concentration that caused maximal hyperalgesia) (FIG. 9) (Pedigo, 1975). While ICV nicotine caused significant analgesia both in the presence and absence of isoflurane, it did not prevent hyperalgesia with isoflurane (FIG. 9). These data suggest that nicotine likely does not prevent isoflurane's hyperalgesia by acting in the brain.

Potential Mechanisms of Isoflurane Hyperalgesia

1. Adrenergic

How could nicotinic inhibition cause hyperalgesia? Nicotine is thought to exert its analgesic action by increasing the release of norepinephrine, serotonin and acetylcholine onto the relay neurons in the dorsal horn of the spinal cord (Rao, 1996; and Reuter, 2000). Isoflurane might reduce pain threshold by inhibiting nicotinic acetylcholine receptors tonically active in potentiating the release of norepinephrine, serotonin and/or acetylcholine. To test the role of the α-adrenergic system, mice were treated systemically with the adrenergic neurotoxin DSP-4 to deplete norepinephrine levels (Jonsson, 1981). The DSP-4 mice 14 days after treatment were tested for HPWL in the presence of isoflurane alone or isoflurane with nicotine pretreatment (see methods). The mice were then sacrificed and the levels of norepinephrine, serotonin, dopamine and their metabolites were measured by HPLC to verify that norepinephrine, but not other transmitters were affected (Table 2).

TABLE 2
HPLC results for Norepinephrine (NE), 5-HT and Dopamine
(DA) concentrations

| Sample Name | NE | MHPG | DA | DOPAC | HVA | 5-HT | 5HIAA |
|---|---|---|---|---|---|---|---|
| Control (n = 2) | | | | | | | |
| Brain Stem | 1044.1 | 253.3 | 599.2 | 662.4 | 300.1 | 2336.9 | 2972.1 |
| Medulla | 1408.8 | 194.5 | 168.7 | 248.3 | 118.5 | 1152.5 | 1184.1 |
| Pons | 1345.9 | 235.9 | 128.1 | 341.8 | 100.7 | 996.8 | 1334.6 |
| Spinal cord | 798.8 | 82.5 | 182.6 | 136.4 | 38.2 | 1122.1 | 1057.9 |
| DSP TrDSP4-Treated (n = 2) | | | | | | | |
| Brain Stem | 811.2 | 104.9 | 621.0 | 494.6 | 275.2 | 2140.3 | 2746.9 |
| Medulla | 812.0 | 139.3 | 138.2 | 196.0 | 88.0 | 1054.9 | 1302.6 |
| Pons | 811.6 | 122.1 | 379.6 | 345.3 | 181.6 | 1597.6 | 2024.7 |
| Spinal cord | 587.6 | 62.6 | 210.9 | 118.8 | 23.0 | 808.6 | 1111.3 |

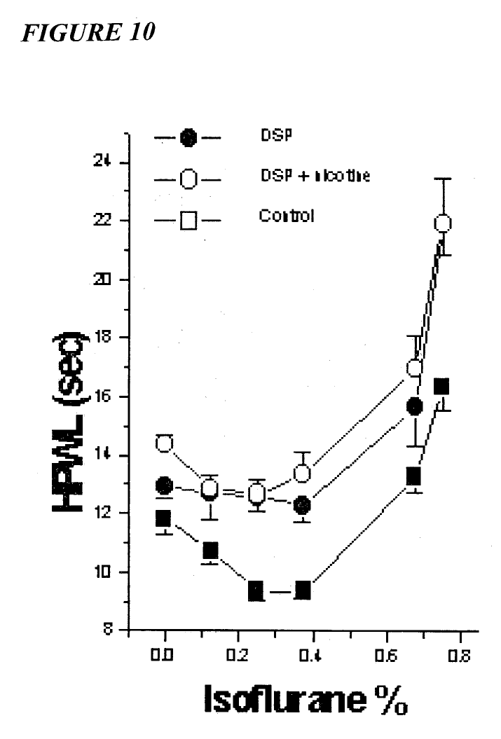

The HPLC procedures were conducted according to the methods described by Underwood et al. (Underwood, 1999). When norepinephrine had been depleted from the brain and spinal cord with DSP-4, isoflurane had no hyperalgesic effect at any subanesthetic concentration. Nicotine was also without effect on the DSP-4 treated mice (FIG. 10).

2. Muscarinic

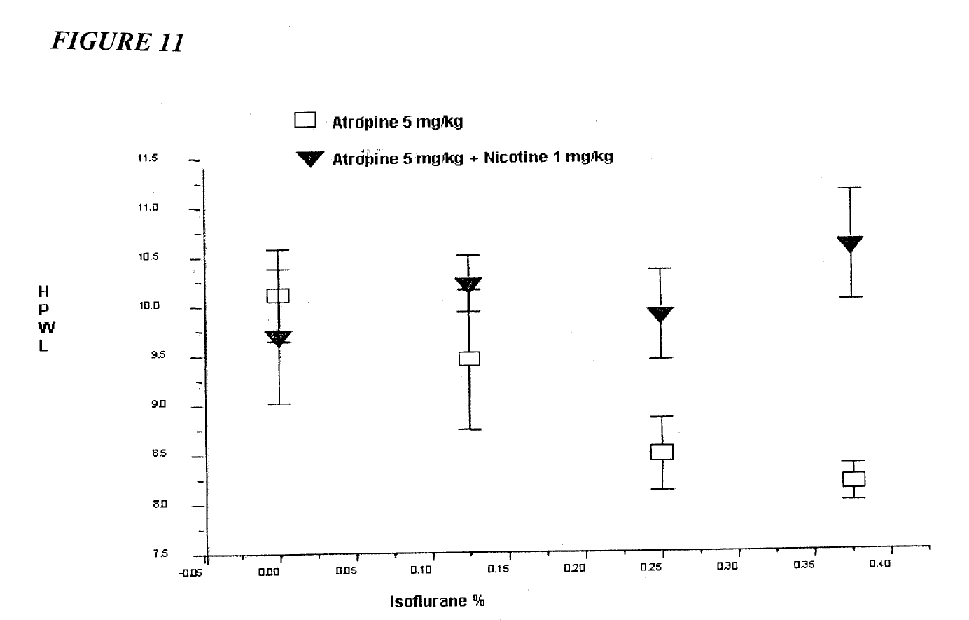

To test the role of muscarinic receptors for acetylcholine, mice were treated with the nonspecific muscarinic antagonist, atropine. A cholinergic neurotoxin could not be used for these studies, because removing all cholinergic neurons would have nicotinic in addition to muscarinic effects. Instead, postsynaptic muscarinic blockade was used. ACh causes analgesia, at least in part due to muscarinic stimulation (Chiari, 1999; Smith, 1989; and Lavand'homme, 1999). The minimum concentration of atropine (5 mg/kg) that caused baseline hyperalgesia was used. High concentrations of atropine can block nicotinic acetylcholine receptors as well (Zwart, 1997). Isoflurane had equal hyperalgesic effects in the presence and absence of atropine (FIGS. 1 and 11). The effect of nicotine on isoflurane hyperalgesia was also unchanged (FIGS. 4 and 11).

Isoflurane Reduces the Nicotine Facilitated Release of Norepinephrine in a Spinal Cord Slice

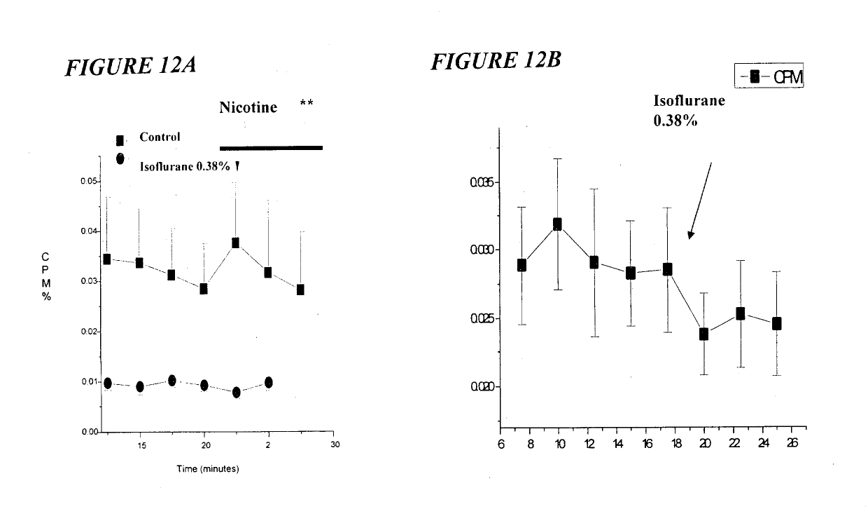

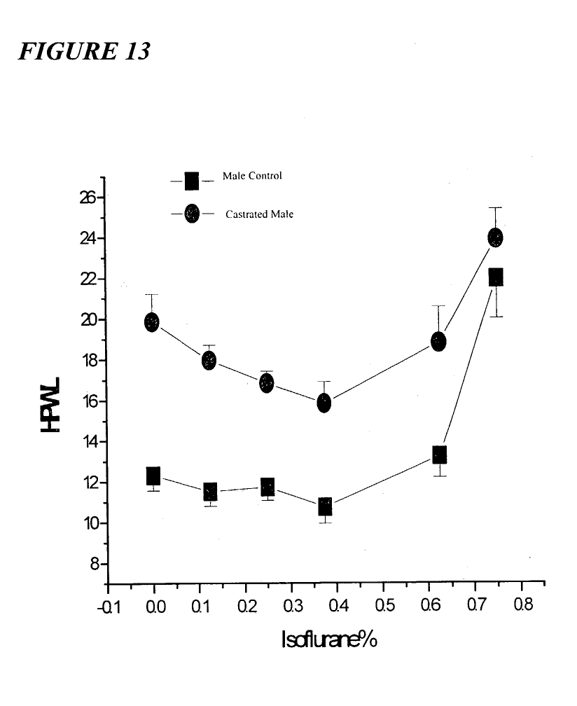

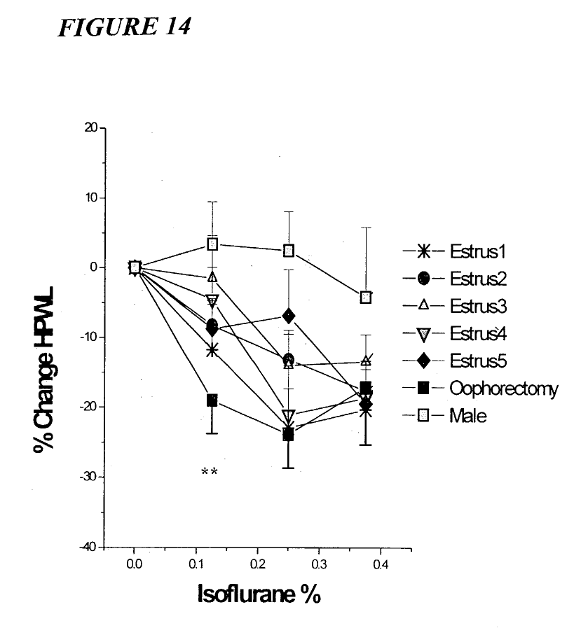

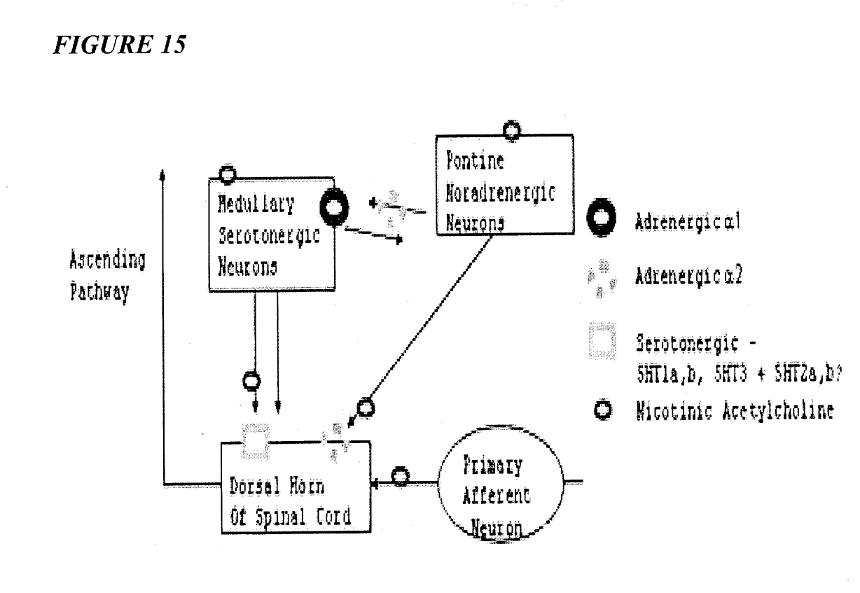

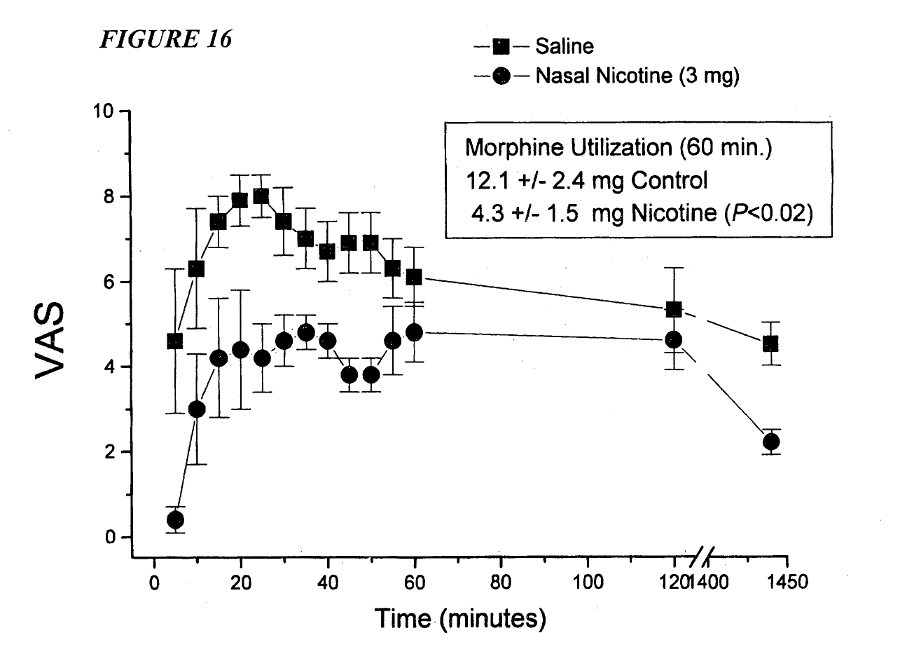

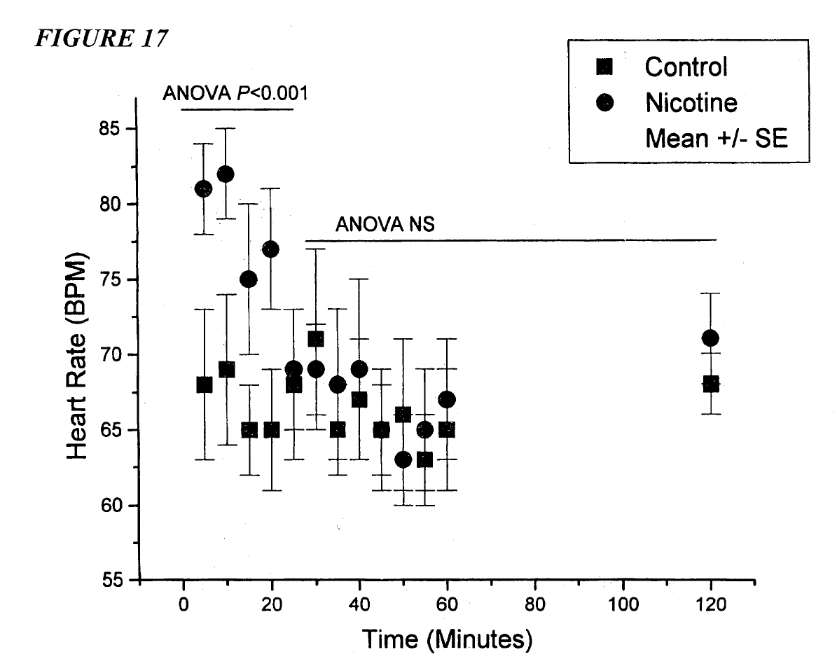

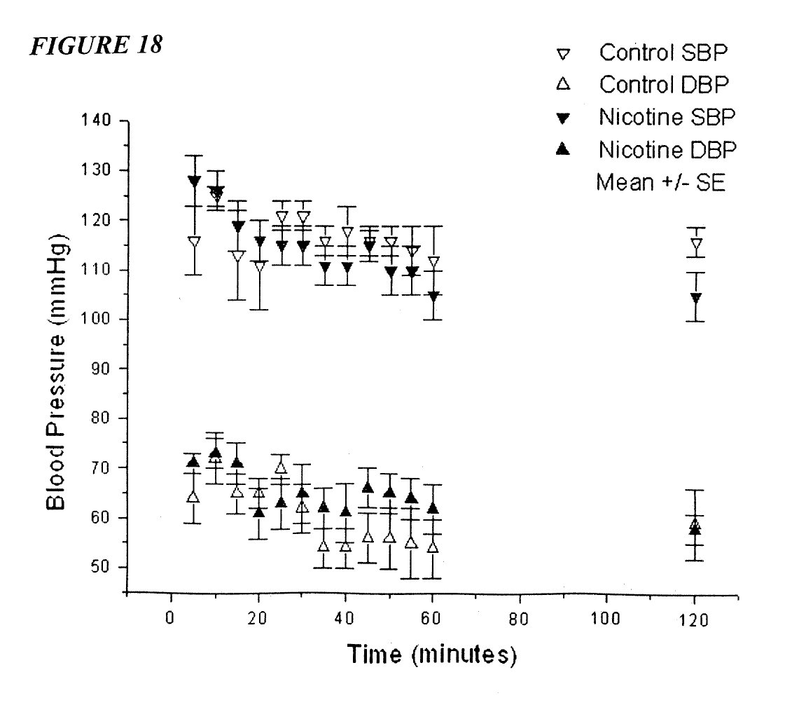

A spinal cord slice preparation prepared from female mouse lumbar spinal cord was used to demonstrate that isoflurane inhibits nicotinic facilitation of norepinephrine release. After incubation with ³H-norepinephrine, the cord slices were perfused with buffer for 30 minutes at 0.5 ml/minute. After equilibration, samples containing released ³H-norepinephrine were collected at 2.5 minute intervals. After determination of baseline release, the slices were stimulated with 1 mM nicotine in the perfusion buffer. Nicotine caused an increase in the release of ³H-norepinephrine from the spinal cord slices that quickly terminated prior to completion of nicotine treatment (FIG. 12A). In the presence of isoflurane (0.38%) basal norepinephrine release was reduced and there was no facilitation by nicotine (FIG. 12B).

Experimental Set III

Gender Differences in Isoflurane Hyperalgesia

Although male mice have some hyperalgesic response to isoflurane it is less than that in females (FIGS. 1 and 13). The difference may be due to the different hormonal milieu. Estrogen can directly modulate nicotinic receptors at micromolar concentrations (Paradiso, 2001; Nakazawa, 2001; and Damaj, 2001). Estrogen receptors are co-localized with both nicotinic and muscarinic receptors (Hosli, 2001). Progesterone is inhibitory at nicotinic acetylcholine receptors (Paradiso, 2001; and Damaj, 2001). HPWL was tested at different phases of the female estrus cycle (FIG. 14). High estrogen (stages 2 and 3) and high progesterone (stage 5) appear to be protective as isoflurane has less hyperalgesic effect during these stages. These findings would suggest that ovarian hormones induce conditions such that isoflurane hyperalgesia is reduced. Oophorectomy (see methods) was performed on six week old female mice. After allowing two weeks for hormonal depletion, and surgical recovery HPWL was again tested. The hyperalgesic response to isoflurane was more pronounced in female mice after oophorectomy, supporting the idea that ovarian hormones induce a physiological state that is resistant to isoflurane hyperalgesia (FIG. 14).

Furthermore, testosterone was tested to see if it induced a state resistant to isoflurane hyperalgesia. Male mice were castrated and after two weeks for surgical recovery and hormone depletion, hyperalgesia in response to isoflurane was tested. Castrated males had increased hyperalgesic responses to isoflurane compared to intact males; supporting the supposition that testosterone induces a state less vulnerable to the hyperalgesic effects of isoflurane (FIG. 13). In addition to the classical “female hormones”, female mice also have testosterone that is made by the ovary (Halling, 1989). Female mice have a peak in testosterone level in mid-cycle that is coincident with the protection from hyperalgesia. Testosterone levels in females decrease after oophorectomy.

Experimental Set IV

In a double blind, randomized trial of women having pelvic surgery with an isoflurane anesthetic, either nicotine (nasal spray, 3 mg) or saline placebo was administered just prior to emergence from anesthesia. Postoperative pain was assessed using the visual analog scale (VAS). In the VAS, patients are asked how much pain they have on a scale from 0-10 where 0 is no pain and 10 is the worst pain imaginable. All patients had access to essentially unlimited morphine in a PCA pump as is routine after this type of surgery. FIG. 16 shows that the patients who received nicotine had significantly lower VAS scores (less pain) than those who received placebo and used a lot less morphine in the first hour (4 mg versus 12 mg) (FIG. 17). There were no severe ill effects. However the patients who received nicotine had higher heart rates. Blood pressures for patients treated with or without nicotine were not different (FIG. 18).

D. Conclusion

Hyperalgesia occurs in vivo at isoflurane doses that antagonize nAChRs in vitro. Since hyperalgesia can be prevented by a nicotinic agonist and can be mimicked and potentiated by nicotinic antagonists, it can be concluded that isoflurane inhibition of nAChRs activation is involved in the pathway that causes hyperalgesia.

References

1. Qian, C., Li, T. Biftu, T. and Shen, T. U.S. Pat. No. 6,077,846.

2. Aceto, M. D., et al. (1986) Neuropharm. 25(9):1031-1036.

3. Armeric et al. (1994) J. Pharmacol. Exp. Ther. 270:310-318.

4. Archer, D. P., et al. (1996) Can. J. Anaesth. 43(5):503-510.

5. Arora, M. V., Carson, I. W. and Dundee, J. W. (1972) Br. J. Anaesth. 44(6):590-592.

6. Badio, B., and Daly, J. W. (1994) Mol. Pharmacol. 45:563-569.

7. Bannon, A. W., et al. (1998) Science 279(5347):77-81.

8. Berkley, K. J. (1997) Pain 70(2-3):103-107.

9. Bitner, R. S., et al. J Neurosci, 1998. 18(14): p. 5426-32.

10. Bovill, J. G. and Dundee, J. W. (1971) Br. J. Anaesth. 43(5):496-499.

11. Briggs, L. P., et al. (1982) Br. J. Anaesth. 54(3):307-311.

12. Cardoso, R. A., et al. (1999) Anesthesiology 91(5):1370-1377.

13. Carla, V. and Moroni, F. (1992) Neurosci. Lett. 146:21-24.

14. Chiari, A., et al. (1999) Anesthesiology 91(5):1447-1454.

15. Christensen, M. K. and Smith, D. F. (1990) J. Neural. Transm. Gen. Sect. 80(3):189-194.

16. Cicero, T. J., Nock, B. and Meyer, E. R. (1996) J. Pharmacol. Exp. Ther. 279(2):767-773.

17. Coates, K. M., et al. (2001) Anesth. Analg. 92(4)930-933.

18. Cordero-Erausquin, M. and Changeux, J. P. (2001) Proc. Natl. Acad. Sci. U.S.A. 98(5):2803-2807.

19. Damaj, M. I., et al. (1998) J. Pharmacol. Exp. Ther. 284(3):1058-1065.

20. Damaj, M. I., Meyer, E. M. and Martin, B. R. (2000) Neuropharmacology 39(13):2785-2791.

21. Damaj, M. I. (2001) J. Pharmacol. Exp. Ther. 296(1):132-140.

22. Dildy-Mayfield, J. E., Eger, E., and Harris, R. A. (1996) J. Pharmacol. Exp. Ther. 276:1058-1065.

23. Downie, D. L., Hall, A. C., Lieb, W. R., and Franks, N. P. (1996) Br. J. Pharmacol. 118:493-502.

24. Dundee, J. and Moore, J. (1960) Br. J. of Anaesth. 32:453-459.

25. Ewen, A., et al. (1995) Can. J. Anaesth. 42(6):532-540.

26. Fields, H. L., Heinricher, M. M. and Mason, P. (1991) Annu. Rev. Neurosci. 14:219-245.

27. Fields, H. and Basbaum, A. (1999) Central Nervous System Mechanisms of Pain Modulation, in Textbook of Pain. P. Wall and R. Melzack, Editors. Churchill Livingstone: Edinburgh. p. 309-330.

28. Fishbein, L., O'Brian, P., et al. (2000) J. Invest. Med. 48(6):435-440.

29. Flood, P., Ramirez-Latorre, J., and Role, L. (1997) Anesthesiology 86(4):859-865.

30. Flood, P. and Role, L. W. (1998) Toxicol. Lett. 100-101:149-153.

31. Flood, P. and Krasowski, M. D. (2000) Anesthesiology, 92(5):1418-1425.

32. Franks, N. and Lieb, W. (1994) Nature 367:607-614.

33. Franks, N. P. and Lieb, W. R. (1998) Toxicol. Lett. 100-101:1-8.

34. Gao, K., et al. (1998) J. Neurosci. 18(5):1860-8.

35. Gao, K. and Mason, P. (2000) J. Neurophysiol. 84(4):1719-1725.

36. Garraway, S. M. and Hochman, S. (2001) J. Neurophysiol. 86(5):2183-2194.

37. Gear, R. W., et al. (1996a) Neurosci. Lett. 205(3):207-209.

38. Gear, R. W., et al. (1996b) Nat. Med. 2(11):1248-1250.

39. Gear, R. W., et al. (1999) Pain 83(2):339-345.

40. Gillberg, P. G., d'Argy, R. and Aquilonius, S. M. (1988) Neurosci. Lett. 90(1-2):197-202.

41. Gillberg, P. G., et al. (1990) Psychopharmacology 100(4):464-469.

42. Guo, T. Z., et al. (1998) Anesthesiology 88(6):1634-1642.

43. Guthrrie, S. K., Zubieta, J. K., et al. (1999) Eur. J. Clin. Pharmacol. 55(9):639-643.

44. Hall, A. C., Lieb, W. R. and Franks, N. P. (1994) Br. J. Pharmacol. 112(3):906-910.

45. Halling, A. and Forsberg, J. G. (1989) J. Steroid Biochem. 32(3):439-443.

46. Harrison, N. L., Kugler, J. L., Jones, M. V., Greenblatt, E. P., and Prichett, D. B. (1993) Mol. Pharmacol. 44:628-632.

47. Hargreaves K. M., Mueller G. P., Dubner R., Goldstein D., and Dionne, R. A. (1987) Brain Res. 422:154-157.

48. Harrison, N. and Flood, P. (1998) Science and Medicine 5:18-27

49. Hosli, E., et al. (2001) Int. J. Dev. Neurosci. 19(1):11-19.

50. Hunt, T. E., Wu, W. H. and Zbuzek, V. K. (1998) Anesth. Analg. 87(5):1109-1112.

51. Iwamoto, E. T. (1991) J. Pharmacol. Exp. Ther. 257(1):120-133.

52. Iwamoto, E. T. and Marion, L. (1993) J. Pharmacol. Exp. Ther. 265(2):777-789.

53. Jacob, P., Wu, S., Yu, L. and Benowitz, N. L. (2000) J. Pharm. Biomed. Anal. 23:653-661.

54. Jonsson, G., et al. (1981) Eur. J. Pharmacol. 72(2-3):173-188.

55. Khan, I. M., et al. (1996) J. Pharmacol. Exp. Ther. 278(1):97-106.

56. Khan, I. M., Taylor, P. and Yaksh, T. L. (1994) J. Pharmacol. Exp. Ther. 271(3):1550-1557.

57. Khan, I. M., et al. (1998) Neuropharmacology 37(12):1515-1525.

58. Kingery, W., et al. (2002) Anesthesiology 96(2):267-274.

59. Lavand'homme, P. M. and Eisenach, J. C. (1999) Anesthesiology 91(5):1455-1461.

60. Li, X., et al. J Neurosci, 1998. 18(5):p. 1904-12.

61. Lin L. H., Chen, L. L., Zirrolli, J. A., and Harris, R. A. (1992) J. Pharmacol. Exp. Ther. 263:569-578.

62. Lloyd G. K., Menzaghi, F., Bontempi, B., et al. (1998) Life Sci. 62:1601-1606.

63. MacDermott, A. B., Role, L. W. and Siegelbaum, S. A. (1999) Annu. Rev. Neurosci. 22:443-485.

64. Marubio, L. M., et al. (1999) Nature 398(6730):805-810.

65. Mattila, M. J., Ahtee, L., and Saarnivaara, L. (1968) Ann. Med. Exp. Biol. Fenn. 46:78-84.

66. McDonough, J. H., Jr. and Shih, T. M. (1995) Pharmacol. Biochem. Behav. 51(2-3):249-253.

67. McGehee, D. S. and Role, L. W. (1995a) Annu. Rev. Physiol. 57:521-546.

68. McGehee, D. S., et al. (1995b) Science 269(5231):1692-1696.

69. Minami, K., Wick, M. J., Stern-Bach, Y., Dildy-Mayfield, J. E., et al. (1998) J. Biol. Chem. 273:8248-8255.

70. Mitchell, S. N. (1993) Neuropharmacology 32(10):937-949.

71. Nakazawa, K. and Ohno, Y. (2001) Eur. J. Pharmacol. 430(2-3):175-183.

72. Nichols, D. E., et al. (1993) Drug Des. Discov. 9(3-4):299-312.

73. North, R. A. and Yoshimura, M. (1984) J. Physiol. 349:43-55.

74. O'Dell, T. J. and Christensen, B. N. (1988) Neurosci. Lett. 94(1-2):93-98.

75. Oyama, T., et al., (1996) Neurosci. Res. 25(2):129-135. 76. Panel, A. P. M. G., Acute Pain Management: Operative or Medical Procedures and Trauma, Clinical Practice Guideline, (1992) Agency for Health Care Policy and Research, Public Health Service, U.S. Department of Health and Human Services: Rockville, Md.

77. Papke, R. L., Sanberg, P. R. and Shytle, R. D; (2001) J. Pharmacol. Exp. Ther. 297(2):646-656.

78. Paradiso, K., et al. (2000) Mol. Pharmacol. 58(2):341-351.

79. Paradiso, K., Zhang, J. and Steinbach, J. H. (2001) J. Neurosci. 21(17):6561-6568.

80. Parvini, S., Hamann, S. R. and Martin, W. R. (1993) J. Pharmacol. Exp. Ther. 265(1):286-293.

81. Pedigo, N. W., Dewey, W. L. and Harris, L. S. (1975) J. Pharmacol. Exp. Ther. 193(3):845-852.

82. Potrebic, S. B., Fields, H. L. and Mason, P. (1994) J. Neurosci. 14(3):1655-1665.

83. Poulain, B., et al. (1987) Brain Res. 435(1-2):63-70.

84. Qian C., Li, T., Shen, T. Y., Libertine-Garahan, L., et al. (1993) Eur. J. Pharmacol. 250:R13-14.

85. Rao, T. S., et al. (1996) Neuropharmacology 35(4):393-405.

86. Ratnakumari, L. and Hemmings, H. C. Jr. (1997) Anesthesiology 86(2):428-439.

87. Reuben, M., Boye, S. and Clarke, P. B. (2000) Eur. J. Pharmacol. 393(1-3):39-49.

88. Rueter, L. E., Meyer, M. D. and Decker, M. W. (2000) Brain Res. 872(1-2):93-101.

89. Sagen, J. and Proudfit, H. K. (1986) Brain Res. 370(1):93-101.

90. Smith, M. D., et al. (1989) Life Sci. 45(14):1255-1261.

91. Tallarida, R. J., Porreca, F., and Cowan, A. (1989) Life Sci. 45:947-961.

92. Tatsuo, M. A., et al. (1997) Braz. J. Med. Biol. Res. 30(2):251-256.

93. Tatsuo, M. A., et al. (1999) Eur. J. Pharmacol. 370(1):9-15.

94. Thompson, J. A., Ho, M. S. and Petersen, D. R. (1982) J. Chromatogr. 231(1):53-63.

95. Uhlen, S. and Wikberg, J. E. (1991) Pharmacol. Toxicol. 69(5):341-350.

96. Underwood, M. D., et al. (1999) Brain Res. 824(1):45-55.

97. Unruh, A. M. (1996) Pain 65(2-3):123-167.

98. Violet, J. M., et al. (1997) Anesthesiology 86(4):866-874.

99. Wakamori, M., Ikemoto, Y. and Akaike, N. (1991) J. Neurophysiol. 66(6):2014-2021.

100. Webster, J. C., Francis, M. M., Porter, J. K., Robinson G., Stokes, C., et al. (1999) Br. J. Pharmacol. 127:1337-1348.

101. Woolf, N. (1991) Prog. Neurobiol. 37:475-524.

102. Zhang, Y., et al. (2000) Anesth. Analg. 91(2):462-466.

103. Zwart, R. and Vijverberg, H. P. (1997) Mol. Pharmacol. 52(5):886-895.

What is claimed is:

1. A method for reducing hyperalgesia following a pain-inducing procedure being performed on a subject which comprises:
(a) administering to the subject an anesthetic which is an antagonist of the subject's nicotinic acetylcholine receptor, in an amount effective to inhibit the subject's perception of pain during the pain-inducing procedure;
(b) then performing the pain-inducing procedure on the subject; and
(c) administering to the subject a hyperalgesia-reducing amount of an agonist of the subject's nicotinic acetylcholine receptor
such that the subject's hyperalgesia following the procedure is reduced.

2. The method of claim 1, wherein steps (b) and (c) occur simultaneously.

3. The method of claim 1, wherein step (c) follows step (b).

4. The method of claim 1, wherein step (c) commences during step (b) and continues after step (b) is performed.

5. The method of claim 1, wherein the subject is a human.

6. The method of claim 1, wherein the subject is a male.

7. The method of claim 1, wherein the subject is a female.

8. The method of claim 7, wherein the female subject has a low level of circulating estrogen.

9. The method of claim 7, wherein the female subject is post-menopausal.

10. The method of claim 1, wherein the hyperalgesia consists of an enhanced sensitivity to pain.

11. The method of claim 1, wherein the pain-inducing procedure is a surgical procedure.

12. The method of claim 1, wherein the anesthetic is isoflurane, halothane, sevoflurane, desflurane, nitrous oxide, ketamine or a barbiturate.

13. The method of claim 1, wherein the nicotinic acetylcholine receptor agonist is administered intranasally.

14. The method of claim 1, wherein the nicotinic acetylcholine receptor agonist is administered transdermally.

15. The method of claim 1, wherein the nicotinic acetylcholine receptor agonist is nicotine.

16. The method of claim 15, wherein the nicotine dose is about 3 milligrams.

17. The method of claim 15, Wherein the nicotine is administered intranasally.

18. The method of claim 16, wherein the nicotine is administered intranasally.

19. The method of claim 1, wherein the nicotinic acetylcholine receptor agonist is administered via a single dose.

20. The method of claim 1, wherein the nicotinic acetylcholine receptor agonist is administered via a plurality of doses.

21. The method of claim 1, wherein the nicotinic acetylcholine receptor agonist is administered while the subject is conscious.

22. The method of claim 1, wherein the nicotinic acetylcholine receptor agonist is administered while the subject is unconscious.

23. The method of claim 1, further comprising administering to the subject during step (c) a pain-reducing amount of a narcotic agent.

24. The method of claim 23, wherein step (b) and step (c) are performed simultaneously.

25. The method of claim 23, wherein step (c) follows step (b).

26. The method of claim 23, wherein step (c) commences during step (b) and continues after step (b) is performed.

27. The method of claim 23, wherein the narcotic agent is morphine, meperidine, or fentanyl.

28. The method of claim 1, further comprising administering to the subject during step (c) a hyperalgesia-reducing amount of a steroid sex hormone.

29. The method of claim 28, wherein step (b) and step (c) are performed simultaneously.

30. The method of claim 28, wherein step (c) follows step (b).

31. The method of claim 28, wherein step (c) commences during step (b) and continues after step (b) is performed.

32. The method of claim 28, further comprising administering to the subject after step (c) a pain-reducing amount of a narcotic agent.

33. The method of claim 28, wherein the sex steroid hormone is a synthetic sex steroid hormone.

34. The method of claim 28, wherein the sex steroid hormone is estrogen.

35. The method of claim 28, wherein the sex steroid hormone is a synthetic estrogen.

36. The method of claim 28, wherein the sex steroid hormone is testosterone.

37. The method of claim 28, wherein the sex steroid hormone is a synthetic testosterone.

38. The method of claim 28, wherein the sex steroid hormone is progesterone.

39. The method of claim 28, wherein the sex steroid hormone is a synthetic progesterone.

40. The method of claim 28, wherein the nicotinic acetylcholine receptor agonist is nicotine.

41. The method of claim 28, wherein the nicotinic acetylcholine receptor agonist is administered intranasally.

42. The method of claim 28, wherein the nicotinic acetylcholine receptor agonist is administered transdermally.

43. The method of claim 28, wherein the nicotinic acetylcholine receptor agonist is administered via a single dose.

44. The method of claim 28, wherein the nicotinic acetylcholine receptor agonist is administered via a plurality of doses.

45. The method of claim 28, wherein the nicotinic acetylcholine receptor agonist is administered while the subject is conscious.

46. The method of claim 28, wherein the nicotinic acetylcholine receptor agonist is administered while the subject is unconscious.

47. An article of manufacture comprising a packaging material having therein a nicotinic acetylcholine receptor agonist and a label indicating a use of the agonist for reducing hyperalgesia in a subject having therein a hyperalgesia-inducing amount of an anesthetic that acts as a nicotinic acetylcholine receptor antagonist.

48. The article of claim 47, wherein the nicotinic acetylcholine receptor agonist is nicotine.

49. An article of manufacture comprising a packaging material having therein a nicotinic acetylcholine receptor agonist and a label indicating a use of the agonist for reducing enhanced pain sensitivity in a subject following surgery on the subject, which surgery employs an anesthetic that acts as a nicotinic acetylcholine receptor antagonist.

50. The article of manufacture of claim 49, wherein the nicotinic acetylcholine receptor agonist is nicotine.
